SEQUENCE LISTING (66 sequences)

SEQ ID NO: 1 - protein, 3 aa, Artificial Sequence
  F8 CDR1 VH
LFT

SEQ ID NO: 2 - protein, 6 aa, Artificial Sequence
  F8 CDR2 VH
SGSGGS

SEQ ID NO: 3 - protein, 6 aa, Artificial Sequence
  F8 CDR3 VH
STHLYL

SEQ ID NO: 4 - protein, 3 aa, Artificial Sequence
  F8 CDR1 VL
MPF

SEQ ID NO: 5 - protein, 7 aa, Artificial Sequence
  F8 CDR2 VL
GASSRAT

SEQ ID NO: 6 - protein, 6 aa, Artificial Sequence
  F8 CDR3 VL
MRGRPP

SEQ ID NO: 7 - protein, 118 aa, Artificial Sequence
  F8 VH domain
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 8 - protein, 108 aa, Artificial Sequence
  F8 VL domain
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 9 - protein, 240 aa, Artificial Sequence
  F8(scFv)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 10 - protein, 231 aa, Artificial Sequence
  F8(Db)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 11 - protein, 118 aa, Artificial Sequence
  KSF VH domain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSS

SEQ ID NO: 12 - protein, 109 aa, Artificial Sequence
  KSF VL domain
SSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLG

SEQ ID NO: 13 - protein, 241 aa, Artificial Sequence
  KSF(scFv)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLG

SEQ ID NO: 14 - protein, 231 aa, Artificial Sequence
  KSF(Db)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGSGGSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLG

SEQ ID NO: 15 - protein, 126 aa, Artificial Sequence
  murine IL9
QRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 16 - protein, 126 aa, Artificial Sequence
  human IL9
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 17 - protein, 626 aa, Artificial Sequence
  F8(scFv)-mIL9-F8(scFv) (“CrAb” format)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSGGSGSGGGGQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRPSGGSGSGGGGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 18 - protein, 626 aa, Artificial Sequence
  F8(scFv)-hIL9-F8(scFv) (“CrAb” format)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSGGSGSGGGGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKISGGSGSGGGGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGGGSGGGGSGGGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 19 - protein, 628 aa, Artificial Sequence
  KSF(scFv)-mIL9-KSF(scFv) (“CrAb” format)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLGSGGSGSGGGGQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRPSGGSGSGGGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLG

SEQ ID NO: 20 - protein, 372 aa, Artificial Sequence
  of murine IL9 conjugated to the C-terminus of the F8 diabody (F8(Db)-mIL9)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGGGGSGGGGSGGGGSQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 21 - protein, 372 aa, Artificial Sequence
  murine IL9 conjugated to the N-terminus of the F8 diabody (mIL9-F8(Db))
QRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRPGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 22 - protein, 372 aa, Artificial Sequence
  human IL9 conjugated to the C-terminus of the F8 diabody (F8(Db)-hIL9)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 23 - protein, 372 aa, Artificial Sequence
  human IL9 conjugated to the N-terminus of the F8 diabody (hIL9-F8(Db))
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 24 - protein, 372 aa, Artificial Sequence
  murine IL9 conjugated to the C-terminus of the KSF diabody (KSF(Db)-mIL9)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSGGSGGSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLGGGGGSGGGGSGGGGSQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 25 - protein, 14 aa, Artificial Sequence
  linker between the VH domain and the VL domain in the F8(scFv) or in the KSF(scFv)
GGGGSGGGGSGGGG

SEQ ID NO: 26 - protein, 5 aa, Artificial Sequence
  the linker between the VH domain and the VL domain in the F8(Db) or in the KSF(Db)
GGSGG

SEQ ID NO: 27 - protein, 15 aa, Artificial Sequence
  linker between murine IL9 or human IL9 and F8 (Db) or KSF(Db)
GGGGSGGGGSGGGGS

SEQ ID NO: 28 - protein, 10 aa, Artificial Sequence
  linker between murine IL9 or human IL9 and the F8(scFv) or the KSF(scFv) in CrAb format
SGGSGSGGGG

SEQ ID NO: 29 - DNA, 65 bp, Artificial Sequence
  Leader sequence-ScFvF8-linker - Forward Primer
tcctcctgttcctcgtcgctgtggctacaggtgtgcactcggaggtgcagctgttggagtctggg

SEQ ID NO: 30 - DNA, 54 bp, Artificial Sequence
  Leader sequence-ScFvF8-linker - Backward Primer
acctccaccgccagaaccacttccgcctgatttgatttccaccttggtcccttg

SEQ ID NO: 31 - DNA, 54 bp, Artificial Sequence
  Linker - hIL9-Linker - Forward Primer
tcaggcggaagtggttctggcggtggaggtcaggggtgtccaaccttggcgggg

SEQ ID NO: 32 - DNA, 32 bp, Artificial Sequence
  Linker - hIL9-Linker - Backward Primer and HindIII-Leader_sequence-ScFvF8-Linker-IL9-Linker - Backward Primer
cccctgaaccactgcctccagatatcttgcct

SEQ ID NO: 33 - DNA, 53 bp, Artificial Sequence
  Linker-mIL9-Linker - Forward Primer
tcaggcggaagtggttctggcggtggaggtcagagatgcagcaccacatgggg

SEQ ID NO: 34 - DNA, 33 bp, Artificial Sequence
  Linker-mIL9-Linker - Backward Primer and HindIII-Leader sequence-ScFvF8-Linker-mIL9-Linker - back ward
ccccctgaaccactgcctccagatggtcggctt

SEQ ID NO: 35 - DNA, 36 bp, Artificial Sequence
  Linker-ScFv8-STOP-NotI - Forward Primer
gcagtggttcagggggcggtggagaggtgcagctgt

SEQ ID NO: 36 - DNA, 42 bp, Artificial Sequence
  Linker-ScFv8-STOP-NotI - Backward Primer and Linker-IL9-Linker-ScFv8-STOP-NotI - Backward Primer
ttttccttttgcggccgcttatttgatttccaccttggtccc

SEQ ID NO: 37 - DNA, 57 bp, Artificial Sequence
  HindIII-Leader_sequence-ScFvF8-Linker-IL9- Linker - Forward Primer
cccaagcttgtcgaccatgggctggagcctgatcctcctgttcctcgtcgctgtggc

SEQ ID NO: 38 - DNA, 25 bp, Artificial Sequence
  Linker-hIL9-Linker-ScFv8-STOP-NotI - Forward Primer
caggggtgtccaaccttggcgggga

SEQ ID NO: 39 - DNA, 24 bp, Artificial Sequence
  Linker-mIL9-Linker-ScFv8-STOP-NotI - Forward Primer
cagagatgcagcaccacatgggga

SEQ ID NO: 40 - DNA, 56 bp, Artificial Sequence
  HindIII-Leader_sequence -DbF8 - Forward Primer; HindIII-Leader_sequence -IL9 - Forward primer; HindIII-Leader_sequence -KSF(VH) - Forward Primer and HindIII-Leader_sequence -DbKSF - forward primer
attaaagcttccaccatgggctggagcctgatcctcctgttcctcgtcgctgtggc

SEQ ID NO: 41 - DNA, 54 bp, Artificial Sequence
  HindIII-Leader_sequence -DbF8 - backward primer
gccagagccacctccgcctgaaccgcctccacctttgatttccaccttggtccc

SEQ ID NO: 42 - DNA, 50 bp, Artificial Sequence
  hIL9-Stop-NotI - Forward primer
caggcggaggtggctctggcggtggcggatcacaggggtgtccaaccttg

SEQ ID NO: 43 - DNA, 39 bp, Artificial Sequence
  hIL9-Stop-NotI - Backward primer
taatgcggccgcttatcatatcttgcctctcatccctct

SEQ ID NO: 44 - DNA, 50 bp, Artificial Sequence
  mIL9-Stop-NotI - Forward primer
caggcggaggtggctctggcggtggcggatcacagagatgcagcaccaca

SEQ ID NO: 45 - DNA, 42 bp, Artificial Sequence
  mIL9-Stop-NotI - Backward Primer
taatgcggccgcttatcatggtcggcttttctgcctttgcat

SEQ ID NO: 46 - DNA, 59 bp, Artificial Sequence
  Leader_sequence-hIL9 - Forward Primer
tcctcctgttcctcgtcgctgtggctacaggtgtgcactcgcaggggtgtccaaccttg

SEQ ID NO: 47 - DNA, 54 bp, Artificial Sequence
  Leader_sequence-hIL9 - Backward Primer
gccagagccacctccgcctgaaccgcctccacctatcttgcctctcatccctct

SEQ ID NO: 48 - DNA, 59 bp, Artificial Sequence
  Leader_sequence-mIL9 - Forward Primer
tcctcctgttcctcgtcgctgtggctacaggtgtgcactcgcagagatgcagcaccaca

SEQ ID NO: 49 - DNA, 57 bp, Artificial Sequence
  Leader_sequence-mIL9 - Backward Primer and HindIII-Leader_sequence -mIL9 - Backward Primer
gccagagccacctccgcctgaaccgcctccacctggtcggcttttctgcctttgcat

SEQ ID NO: 50 - DNA, 53 bp, Artificial Sequence
  DbF8-Stop-NotI - Forward Primer
caggcggaggtggctctggcggtggcggatcagaggtgcagctgttggagtct

SEQ ID NO: 51 - DNA, 39 bp, Artificial Sequence
  DbF8-Stop-NotI - Backward Primer
taatgcggccgcttatcatttgatttccaccttggtccc

SEQ ID NO: 52 - DNA, 56 bp, Artificial Sequence
  HindIII-Leader_sequence -KSF(VH) - Backward Primer
accgccagagccacctccgcctgaaccgcctccaccactcgagacggtgaccaggg

SEQ ID NO: 53 - DNA, 57 bp, Artificial Sequence
  KSF(VL)-linker - Forward Primer
aggcggttcaggcggaggtggctctggcggtggcggatcgtctgagctgactcagga

SEQ ID NO: 54 - DNA, 56 bp, Artificial Sequence
  KSF(VL)-linker - Backward Primer
ctctgacctccaccgccagaaccacttccgcctgagcctaggacggtcagcttggt

SEQ ID NO: 55 - DNA, 34 bp, Artificial Sequence
  linker-mIL9-KSF(ScFv)-NotI - Forward Primer
atggggaattcgagacaccaattaccttattgaa

SEQ ID NO: 56 - DNA, 36 bp, Artificial Sequence
  linker-mIL9-KSF(ScFv)-NotI - Backward Primer
taatgcggccgcttatcatcccagcactgtcagctt

SEQ ID NO: 57 - DNA, 84 bp, Artificial Sequence
  HindIII-Leader_sequence -DbKSF - Backward Primer
aatcggctggctgtggtattcggcggagggaccaagctgaccgtcctaggcggtggaggcggttcaggcggaggtggctctggc

SEQ ID NO: 58 - DNA, 38 bp, Artificial Sequence
  Fragment-A sequence - Forward Primer
tttgactactggggccagggaaccctggtcaccgtctc

SEQ ID NO: 59 - DNA, 63 bp, Artificial Sequence
  Fragment-A sequence - Backward Primer
ccaccgccagagccacctccgcctgaaccgcctccaccacccagagacagggagaggctcttc

SEQ ID NO: 60 - protein, 444 aa, Artificial Sequence
  Amino acid sequence of the F8 heavy chain
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 215 aa, Artificial Sequence
  Amino acid sequence of the F8 light chain in the F8 (IgG4-HC)-mIL9 and F8 (IgG4-HC)-hIL9 conjugates
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 585 aa, Artificial Sequence
  Amino acid sequence of F8 heavy chain-mIL9 in the F8 (IgG4-HC)-mIL9 conjugate
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 63 - protein, 585 aa, Artificial Sequence
  Amino acid sequence of F8 heavy chain-huIL9 in the F8 (IgG4-HC)-hIL9 conjugate
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 64 - protein, 214 aa, Artificial Sequence
  Amino acid sequence of KSF light chain in KSF (IgG4-HC)-mIL9 and KSF (IgG4-HC)-hIL9 conjugates
SSELTQDPAVSVALGQTVRITCQGDSLRSYYASWYQQKPGQAPVLVIYGKNNRPSGIPDRFSGSSSGNTASLTITGAQAEDEADYYCNSSPLNRLAVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 65 - protein, 585 aa, Artificial Sequence
  Amino acid sequence of KSF heavy chain-mIL9 in the KSF (IgG4-HC)-mIL9 conjugate
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 66 - protein, 585 aa, Artificial Sequence
  Amino acid sequence of KSF heavy chain-hIL9 in the KSF (IgG4-HC)-hIL9 conjugate
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPKVSLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

(19) United States
(12) Patent Application Publication
(10) Pub. No.: US 2023/0285585 A1
(43) Pub. Date: Sep. 14, 2023

(54) TREATMENT OF PULMONARY HYPERTENSION

(71) Applicant: PHILOGEN S.P.A., Siena (IT)

(72) Inventors: Alessandra Micaela VILLA, Otelfingen (CH); Mattia MATASCI, Otelfingen (CH); Baptiste GOUYOU, Otelfingen (CH); Anne KERSCHENMEYER, Otelfingen (CH)

(73) Assignee: PHILOGEN S.P.A., Siena (IT)

(21) Appl. No.: 18/006,274

(22) PCT Filed: Jul. 21, 2021
(86) PCT No.: PCT/EP2021/070357

§ 371 Date: Jan. 20, 2023

(30) Foreign Application Priority Data
Jul. 22, 2020 (EP) 20187278.5
Jan. 8, 2021 (EP) 21150760.3

(51) Int. Cl. A61K 47/68; A61P 9/12; A61P 11/00

(52) U.S. Cl. CPC A61K 47/6813; A61K 47/6843; A61P 9/12; A61P 11/00

(57) ABSTRACT

The present application relates to the treatment of pulmonary hypertension (PH) using interleukin-9 (IL9) and particularly, although not exclusively, to the treatment of PH using IL9 conjugated to a specific binding member that binds an antigen associated with tissue and/or vascular remodelling, such as the Extra Domain-A (ED-A) of fibronectin. Conjugates comprising a specific binding member that binds an antigen associated with tissue and/or vascular remodelling, such as ED-A, and IL9 that are suitable for the treatment of PH, in particular conjugates in which IL9 is conjugated to a single-chain Fv that binds ED-A or in which IL9 is conjugated an IgG that binds ED-A, are also disclosed.

### RELATED APPLICATIONS

The present case is related to EP20187278.5 filed on 22 Jul. 2020 and EP21150760.3 filed on 8 Jan. 2021, the contents of both of which are hereby incorporated by reference in their entirety.

### FIELD OF THE INVENTION

The present invention relates to treatment of pulmonary hypertension (PH) using interleukin-9 (IL9) and particularly, although not exclusively, to the treatment of PH using IL9 conjugated to a specific binding member that binds an antigen associated with tissue and/or vascular remodelling, such as the Extra Domain-A (ED-A) of fibronectin. Conjugates comprising a specific binding member that binds an antigen associated with tissue and/or vascular remodelling, such as ED-A, and IL9 that are suitable for the treatment of PH, in particular a conjugate in which IL9 is conjugated to a single-chain Fv that binds ED-A, or a conjugate in which IL9 is conjugated to an IgG that binds ED-A, also form part of the invention.

### BACKGROUND

Pulmonary hypertension (PH) is a pathophysiological disorder that may involve multiple clinical conditions and is a complication of the majority of cardiovascular and respiratory diseases. PH is a disease defined as an increase in mean pulmonary arterial pressure (PAPm) 25 mmHg at rest, as assessed by right heart catheterization (RHC). PH can be divided into five main groups: pulmonary arterial hypertension (“PAH”) (Group 1), PH due to left heart disease (“LHD”) (Group 2), PH due to lung diseases and/or hypoxaemia (Group 3), PH due to chronic pulmonary arterial (PA) obstruction (Group 4), and PH with unclear and/or multifactorial mechanisms (Group 5) (Galiè N. et al. “2015 ESC/ERS Guidelines for the diagnosis and treatment of pulmonary hypertension—web addenda” (2016) European Heart Journal, Volume 37, issue 1, pages 67-119). Current treatment options for PH in general act by decreasing vascular tone and thereby reducing pulmonary artery pressure. Most currently available treatment options are approved for PAH (Group 1), a rare disease, and are therefore not suitable for the treatment of the majority of patients suffering from other, more common, forms of PH as mentioned above.

Interleukin-9 (IL9) is an abundant cytokine mainly secreted by CD4+ T cells after stimulation by transforming growth factor beta (TGFB) and IL4. IL9 signalling is mediated by specific IL9 receptor α-chain dimerized with the common γ-chain cytokine receptor (common to IL2). IL9 has been shown to activate T cells, eosinophils, type 2 innate lymphoid cells (ILC2) and mast cells.

Overexpression of IL9 and its receptor in lung biopsies of patients is correlated with an asthma phenotype and fibrotic condition of patient lungs (Shimbara et al., J. Allergy Clin. Immunol. 2000, 105, 108-115; Sugimoto et al., Am. J. Respir. Cell Mol. Biol. 2019, 60, 232-243). IL9 has mainly been described to induce especially allergic airway inflammation. Here, natural killer and T_{H}9 cells could be identified as the main source of IL9 leading to mast cell degranulation and thereby promoting allergic inflammation (Noelle & Nowak Nat Rev Immunol. 2010; 10:683-7); (Sitkauskiene et al Respir Res. 2005; 6:33) (Cheng, et al., Am J Respir Crit Care Med. 2002; 166:409-16; Jones et al. J Immunol. 2009; 183(8):5251-60).

Interestingly, besides airway inflammation, a similar action of IL9 could also be proven for oral antigen-induced anaphylaxis promoting mucosal inflammation with throat swelling and associated symptoms (Osterfeld et al J Allergy Clin Immunol. 2010; 125:469-76 e2). The expression of IL-9 in the airway epithelium of transgenic mice has been shown to result in airway inflammation, mast cell hyperplasia, and to dramatically increases airway hyperresponsiveness (Townsend et al. Immunity. 2000; 13:573-83).

In response to microbial colonization of the lung, IL9 has been reported to promote cystic fibrotic pathways by creating an inflammation loop via mast cells and ILC2 and the production of IL2 and IL9 (Moretti et al., Nat Commun., 2017, 8, 14017). Constitutive overexpression of IL9 in transgenic mice results in a pathological lung morphology change to an airway inflammation phenotype: increase in the thickening of blood vessels walls, deposition of collagen, lung hypersensitiveness, over expression of inflammation mediators (e.g. IL13 and histamine) and increase number of lung eosinophils (Temann et al., Int. Immunol., 2007, 19, 1-10; Temann et al., J. Clin. Invest., 2002, 109, 29-39; Temann et al., J. Exp. Med., 1998, 188, 1307-1320; Whittaker et al., Am. J. Respir. Cell Mol. Biol., 2002, 27, 593-602; Wilhelm et al., Nat. Immunol., 2011, 12, 1071-1077). Antibody mediated neutralization of IL9 has been shown to improve microbial lung fibrosis and inflammation phenotype, decrease lung inflammation and lung tissue damage caused by oxidative stress in a murine model of chronic obstructive pulmonary disease (Zou et al., Eur Rev Med Pharmacol Sci. 2018, 22, 8877-8884) and suppress lung inflammation and fibrosis phenotype in silica intranasally treated mice (e.g. decreased IL6, ID 2 and TNFα levels) (Sugimoto et al., Am. J. Respir. Cell Mol. Biol. 2019, 60, 232-243). Furthermore, it has been suggested that IL-9 may participate in the process of Ang-II induced hypertension (Yang et al., Mediators of Inflammation 2020 Article ID 5741047). Taken together this data strongly suggests that IL9 promotes lung inflammation and fibrosis in different murine models.

The present invention has been devised in light of the above considerations.

### SUMMARY OF THE INVENTION

The present inventors have shown that conjugates comprising IL9 and an anti-EDA binding member improved symptoms of PH in a mouse model of PH. Specifically, an attenuation of both the right ventricular systolic pressure, as well as surrogate markers of right ventricular load, as assessed by echocardiography, was observed. In contrast, administration of an identical conjugate comprising a binding member to an irrelevant antigen did not improve symptoms in the same mouse model, showing that administration of untargeted IL9 did not have therapeutic efficacy (FIGS. 6 to, 8, 9A-9B, 11 and 13 to 16).

This was highly surprising considering the prior art teachings, which suggested that IL9 worsens a number of lung diseases by promoting inflammatory responses. Inflammatory and autoimmune processes are crucially involved in pathogenesis of both PAH (Group 1) and also PH due to left heart or lung disease (Group 2 and 3).

The majority of currently approved treatments are for the treatment of PAH (Group 1), which is rare condition. These treatments generally act by decreasing vascular tone and thereby reducing pulmonary artery pressure. Common treatments for PAH include phosphodiesterase 5 inhibitors, endothelin receptor antagonists (such as Macitentan), soluble guanylate cyclase stimulators and prostanoids. PH due to left heart or lung disease (Group 2 and 3) are common secondary conditions resulting from a primary lung or heart condition, such as chronic obstructive pulmonary disease or pulmonary fibrosis, and treatment is usually focused on the treatment of the primary condition underlying the disease. PH due to chronic PA obstruction (Group 4) is usually treated through the administration of anti-coagulants if the obstruction is caused by blood clots. Where the obstruction is caused by scar tissue, a pulmonary endarterectomy or balloon pulmonary angioplasty may be performed to improve blood flow and reduce pressure inside the arteries. Soluble guanylate cyclase stimulator or another pulmonary vasodilator may be administered after surgery. Due to the diverse factors underlying the disease, there is no standardised treatment for PH with unclear and/or multifactorial mechanisms (Group 5).

The ED-A of fibronectin is known to be deposited in the extra-cellular matrix (ECM) during tissue remodelling and angiogenesis and expression of ED-A has been reported in lung tissue in spatial association to vessel structures, and to a lesser extent in the lung parenchymal and stromal compartment, in a rat model of PH (Franz et al., Oncotarget, 2016, 7, 81241-81254). The ED-A is also known to be expressed in the remodelled right ventricular myocardium in PH patients. The ED-A of fibronectin, as well as other components of PH-associated tissue remodelling, may therefore be used as antigens for the targeted delivery of IL9 to sites of disease in PH patients. Such targeted delivery is expected to be suitable for the treatment of PH, independent of the underlying reason for the PH.

The present invention thus relates to the targeted delivery of IL9 to sites of disease in patients with pulmonary hypertension, such as remodelled lung and/or heart tissue and/or remodeled vasculature in the heart and/or lung of PH patients. Examples of remodelled lung tissue in PH patients includes in particular remodelled lung tissue in spatial association to vessel structures, as well as remodelled lung tissue in the lung parenchymal and stromal compartment. Remodelled heart tissue in PH patients includes the remodelled right ventricular myocardium.

In one embodiment, the present invention relates to a conjugate for use in a method for treatment of pulmonary hypertension in a patient, the conjugate comprising interleukin-9 (IL9) and a specific binding member which binds an antigen associated with tissue and/or vascular remodelling.

Also provided is a conjugate for use in a method of delivering IL9 to sites of remodelled lung and/or heart tissue or vasculature in a patient with pulmonary hypertension, the conjugate comprising IL9 and a specific binding member which binds an antigen associated with tissue and/or vascular remodelling.

Further provided is a method of treating pulmonary hypertension in a patient, the method comprising administering to the patient a therapeutically effective amount of a conjugate comprising IL9 and a specific binding member which binds an antigen associated with tissue and/or vascular remodelling.

Yet further provided is a method of delivering IL9 to sites of remodelled lung and/or heart tissue or vasculature in a patient with pulmonary hypertension, the method comprising administering a conjugate comprising IL9 and a specific binding member which binds an antigen associated with tissue and/or vascular remodelling to the patient.

The present invention also provides the use of a conjugate comprising IL9 and a specific binding member which binds an antigen associated with tissue and/or vascular remodelling in the manufacture of a medicament for use in a method of treating pulmonary hypertension in a patient.

Further provided is the use of a conjugate comprising IL9 and a specific binding member which binds an antigen associated with tissue and/or vascular remodelling in the manufacture of a medicament for use in a method of delivering IL9 to sites of remodelled lung and/or heart tissue or vasculature in a patient with pulmonary hypertension.

The specific binding member is preferably an antibody molecule or an antigen-binding fragment thereof. For example, the specific binding member may be an immunoglobulin G (IgG) molecule, in particular IgG4, or an antigen-binding fragment thereof. Antigen-binding fragments of antibody molecules are known and include, for example, single-chain Fvs (scFvs), single-chain diabodies, and diabodies.

The specific binding member may comprise a single specific binding member or more than one specific binding members, e.g. two specific binding members, such as two scFvs. Where the conjugate comprises more than one specific binding member, the specific binding members may be the same or different but preferably are the same. The IL9 may be conjugated to the N- or C-terminus of the specific binding member. Where the conjugate comprises two specific binding members, the specific binding member is preferably conjugated to the N-terminus and C-terminus of IL9.

In a preferred embodiment, the specific binding member binds to the ED-A of fibronectin. The specific binding member preferably comprises an antigen-binding site having the complementarity determining regions (CDRs) of antibody F8 set forth in SEQ ID NOs 1 to 6. The antigen binding site may comprise VH and/or VL domains of antibody F8 set forth in SEQ ID NOs 7 and 8, respectively. The specific binding member may comprise or consist of the F8 single-chain Fv amino acid sequence set forth in SEQ ID NO: 9. In one preferred embodiment, the specific binding member comprises or consists of the F8 IgG4 heavy and light chain amino acid sequences set forth in SEQ ID NOs: 60 and 61 respectively.

Other antibodies capable of binding to the ED-A of fibronectin are known, or may be prepared by those skilled in the art, and such antibodies, or antigen-binding fragments of such antibodies, for example their CDRs, VH and/or VL domains, may be used in specific binding members for use in the present invention.

The present inventors have shown that a conjugate in which IL9 was conjugate to antibody F8 in scFv format at the N- and C-terminus of IL9 (conjugate F8(scFv)-mIL9-F8(scFv)) exhibited a 3° C. increased melting temperature compared with conjugates in which IL9 was conjugated to N- or C-terminus of antibody F8 in diabody format (conjugates mIL9-F8(db) and F8(db)-mIL9; FIG. 5A). The F8(scFv)-mIL9-F8(scFv) conjugate also exhibited good thermal stability over time when stored at 37° C. (FIG. 5B) or 4° (FIG. 5C) for a period of up to 7 days or when subjected to 3 cycles of freeze and thawing (FIG. 5D), with no loss of concentration, or changes in the size exclusion profile or SDS-PAGE analysis being detected.

The F8(scFv)-mIL9-F8(scFv) conjugate was further shown to significantly reduce pressure values in the right ventricle (the main pathophysiological surrogate of PH) and significantly improved the majority of echocardiographic signs of right ventricular load and dysfunction in a mouse model of PH compared with the administration of untargeted IL9 (conjugate KSF(scFv)-mIL9-KSF(scFv)). The latter are known to determine prognosis in humans suffering from PH. The effects of F8(scFv)-mIL9-F8(scFv) treatment were at least comparable to the effects achieved by MACI, an established standard therapy for PHA in humans (FIGS. 6, 9A, 9B, to 11).

The present inventors have further shown that a conjugate in which two IL9 moieties were conjugate via their N-termini to the C-termini of the two heavy chains of F8 in IgG4 format (IgG-HC4mIL9) significantly reduce pressure values in the right ventricle and significantly improved a variety of echocardiographic signs of right ventricular load and dysfunction in a mouse model of PH compared with the administration of untargeted IL9. The latter are known to determine prognosis in humans suffering from PH. The effects of IgG-HC4mIL9 treatment were improved compared with the effects achieved by MACI, an established standard therapy for PHA in humans (FIGS. 13 to 16).

IL9-containing conjugates based on the anti-ED-A F8 antibody have been previously prepared and tested for therapeutic efficacy. In particular, a conjugate comprising two murine IL9 (mIL9) moieties conjugated to the N- and C-terminus of antibody F8 in diabody format was shown to be capable of selectively targeting tumors but did not show any significant anti-tumor activity in an F9 teratocarcinoma model (Venetz et al., PNAS, 2015; 112(7):2000-2005; Venetz 2016, Engineered cytokine derivatives for targeted cancer immunotherapy, Doctoral Thesis, ETH Zurich). Two other conjugates comprising F8 and mIL9 of undisclosed format were similarly shown to selective target tumors and one of said conjugates further showed tumour growth retardation in a CT26 colon carcinoma model but no significant therapeutic efficacy in an F9 teratocarcinoma model or in a model of rheumatoid arthritis (Venetz et al., PNAS, 2015; 112(7):2000-2005; Gouyou et al. Interleukin-9 targeted delivery: preclinical efficacy evaluation in cancer and rheumatoid arthritis. Poster presented at: Festival of Biologics in Basel; 2019 Oct. 15-17).

However, a conjugate in which IL9 was conjugated via its N-terminus and C-terminus to two specific binding members has not been previously described.

Thus, in a second aspect, the present invention relates to a conjugate comprising interleukin-9 (IL9), wherein the N-terminus of IL9 is conjugated to a first specific binding member which binds an antigen associated with tissue and/or vascular remodelling and the C-terminus of IL9 is conjugated to a second specific binding member which binds an antigen associated with tissue and/or vascular remodelling. The antigen associated with tissue and/or vascular remodelling bound by the first and second scFvs is preferably the same. The specific binding members may comprise or consist of single chain Fvs (scFvs), diabodies or single chain diabodies, but preferably consist of scFvs.

Thus, in a preferred embodiment, the present invention relates to a conjugate comprising interleukin-9 (IL9), wherein the N-terminus of IL9 is conjugated to a first single-chain Fv (scFv) which binds an antigen associated with tissue and/or vascular remodelling and the C-terminus of IL9 is conjugated to a second scFv which binds an antigen associated with tissue and/or vascular remodelling. The antigen associated with tissue and/or vascular remodelling bound by the first and second scFvs is preferably the same.

The two scFvs are preferably conjugated to the N- and C-termini of the IL9 via amino acid linkers. The linkers may be any suitable length but preferably are 10 to 20 amino acids long. In a preferred embodiment, the amino acid linkers comprise, or consist of, the amino acid sequence set forth in SEQ ID NO: 28.

To our knowledge, a conjugate in which IL9 was conjugated via its N-terminus to the C-terminus of the heavy chain of an immunoglobulin has also not been previously described.

Thus, in a further aspect, the present invention relates to a conjugate comprising interleukin-9 (IL9), wherein the N-terminus of IL9 is conjugated to the C-terminus of the heavy chain of an immunoglobulin molecule which binds an antigen associated with tissue and/or vascular remodelling. In one embodiment, the C-terminus of one of the two heavy chains of the immunoglobulin molecule is conjugated to the N-terminus of an IL9 moiety, so that the conjugate comprises one IL9 moiety. In this embodiment, the C-terminus of the second heavy chain is preferable free. Free” in this context refers to the C-terminus of the heavy chain not being linked or otherwise conjugated to another moiety, such as IL9. In a preferred embodiment, however, the C-termini of the two heavy chains of the immunoglobulin molecule are each conjugated to the N-terminus of an IL9 moiety, so that the conjugate comprises two IL9 moieties. IL9 is preferably conjugated to the C-terminus/i of the heavy chain(s) via amino acid linkers. The linkers may be any suitable length but preferably are 10 to 20 amino acids long. In a preferred embodiment, the amino acid linkers comprise, or consist of, the amino acid sequence set forth in SEQ ID NO: 27.

In a preferred embodiment, the antigen associated with tissue and/or vascular remodelling is the Extra Domain-A of fibronectin. Specific binding members, such as antibodies and antigen-binding fragments thereof, which bind the ED-A of fibronectin are known and include the antibody F8.

In a preferred embodiment, the antibody or antigen-binding fragment thereof, e.g. IgG or scFv, comprises an antigen-binding site having the complementarity determining regions (CDRs) of antibody F8 set forth in SEQ ID NOs 1-6.

In a more preferred embodiment, the antibody or antigen-binding fragment thereof, e.g. IgG or scFv, comprises the VH and VL domains of antibody F8 set forth in SEQ ID NOs 7 and 8.

Most preferably, the scFv comprises the amino acid sequence of scFv F8 set forth in SEQ ID NO: 9.

In an alternative most preferred embodiment, the immunoglobulin molecule, e.g. IgG, comprises the heavy chains and light chains of antibody F8 set forth in SEQ ID NOs 60 and 61.

The IL9 is preferably human IL9 and may comprise, or consist of the amino acid sequence set forth in SEQ ID NO: 16.

In a preferred embodiment, the conjugate has at least 70% sequence identity, more preferably one of at least 75%, 80%, 85%, 90%, 95%, 96%, 97%, 98%, 99% or 100%, sequence identity, to the amino acid sequence of conjugate set forth in SEQ ID NO: 18 (F8(scFv)-hIL9-F8(scFv)). Yet more preferably, the conjugate comprises or consists of the amino acid sequence set forth in SEQ ID NO: 18.

In a further preferred embodiment, the conjugate comprises an IgG molecule, wherein the heavy and light chains of the IgG molecule have at least 70% sequence identity, more preferably one of at least 75%, 80%, 85%, 90%, 95%, 96%, 97%, 98%, 99% or 100%, sequence identity, to the amino acid sequences set forth in SEQ ID NOs 63 and 61, respectively.

The conjugate of the invention may be for use in a method for treatment of the human body by therapy.

The conjugate of the invention is preferably for use in a method for treatment of pulmonary hypertension in a patient. Alternatively, the conjugate of the invention is for use in a method of delivering IL9 to sites of remodelled lung and/or heart tissue or vasculature in a patient with pulmonary hypertension.

The present invention also provides a method of treating pulmonary hypertension in a patient, the method comprising administering to the patient a therapeutically effective amount of a conjugate of the invention. Further provided is a method of delivering IL9 to sites of remodelled lung and/or heart tissue or vasculature in a patient with pulmonary hypertension, the method comprising administering a conjugate of the invention to the patient.

Also provided is the use of a conjugate of the invention in the manufacture of a medicament for use in a method of treating pulmonary hypertension in a patient. Yet further provides is the use of a conjugate of the invention in the manufacture of a medicament for use in a method of delivering IL9 to sites of remodelled lung and/or heart tissue or vasculature in a patient with pulmonary hypertension.

The present invention also provides a nucleic acid molecule encoding a conjugate of the invention. An expression vector comprising such a nucleic acid is similarly provided, as is a host cell comprising such a nucleic acid or expression vector.

Also provided is method of producing a conjugate of the invention, the method comprising culturing a host comprising a nucleic acid or expression vector encoding a conjugate of the invention under conditions for expression of the conjugate, the method optionally further comprising isolating and/or purifying the conjugate following expression.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIGS. 1A-1C: Immunocytokines containing IL9 (human or murine) as payload were cloned by PCR assembly. The F8 diabody and the negative control KSF diabody were genetically fused to the N-terminus of IL9 (FIG. 1A) or to the C-terminus of IL9 (FIG. 1B). An immunocytokine in the “CrAb” format was also prepared with the IL9 gene fused between two F8 scFv's (C). SP=signal peptide, VH=variable heavy chain, VL=variable light chain.

FIGS. 2A-2C: Human IL9 and murine IL9 immunocytokines were successfully expressed by transient gene expression (TGE) in Chinese hamster ovary (CHO) cells. The purified immunocytokines exhibited good biochemical properties as confirmed using SDS-PAGE and size exclusion chromatography. On SDS-PAGE, the immunocytokines showed bands at around 50 kDa for the F8 diabody based fusions (FIGS. 2A-2B) or 75 kDa for the immunocytokines in F8 CrAb format (FIG. 2C) and in KSF CrAb format (FIG. 2D). These values were slightly higher than the estimated sizes of about 42 kDa and 65 KDa for the diabody and CrAb based immunocytokines, respectively. The shift was most likely caused by the presence of 4 N-glycosylation sites within IL9. The presence of single peaks in SEC analysis confirmed the homogeneity of the immunocytokine preparations.

FIGS. 3A-3F: shows binding of the purified (A) mIL9-F8(db), (B) hIL9-F8(db), (C) F8(db)-mIL9, (D) F8(db)-hIL9, (E) F8(scFv)-mIL9-F8(scFv) and (F) F8(scFv)-hIL9-F8(scFv) immunocytokines to EDA as measured by surface plasmon resonance (SPR). SPR analysis showed comparable apparent KD for all fusion proteins, in the nM range.

FIG. 4 shows in vitro bioactivity characterization of mIL9 based immunocytokines. Bioactivity of the murine IL9 immunocytokines was assessed by the ability of the immunocytokines to stimulate the proliferation of MC/9 cells. Although the activity of the immunocytokines appeared lower than the activity of a commercial mIL9 preparation used as a positive control, all of the various mIL9 immunocytokines showed biological activities with EC50 values in the range of 0.02 to 0.07 nM for F8(scFv)-mIL9-F8(scFv) and F8(db)-mIL9 and 0.012 to 0.034 nM for mIL9-F8(db).

FIGS. 5A-5D: Stability characterization of mIL9 immunocytokines proteins. The “Crab” format F8(scFv)-mIL9-F8(scFv) displayed approximately a 3° C. increased melting temperature compared to the two immunocytokines in diabody formats F8(db)-mIL9 and mIL9-F8(db) (FIG. 5A). F8(scFv)-mIL9-F8(scFv) also displayed good thermal stability over time with regard to loss of protein concentration, aggregation, or degradation as assessed by i) OD280 measurement, ii) SEC analysis, and iii) SDS-PAGE analysis. Indeed, when incubated at 37° C. (FIG. 5B) or 4° (FIG. 5C) for a period of up to 7 days or when tested over 3 cycles of freeze and thawing (FIG. 5D), no loss of concentration, no changes in the size exclusion profile or SDS-PAGE analysis were detected. Taken together, these results suggest that the F8(scFv)-mIL9-F8(scFv) immunocytokine has improved stability compared with the diabody-based immunocytokines.

FIG. 6 shows the experimental design of the monocrotaline (MCT)-induced pulmonary hypertension (PH) mouse model and treatment schedule used to assess the potential beneficial effects of a targeted delivery of Interleukin-9 (IL9) using the human recombinant antibody F8 specific to the alternatively spliced ED-A domain of fibronectin (ED-A⁺ Fn). The following experimental groups were analyzed:
Sham induced controls (no PH) (n=6)
MCT induced PH (n=8)
MCT induced PH+macitentan (MACI) (n=7)
MCT induced PH+F8(scFv)-mIL9-F8(scFv) (F8-IL9) (n=6)
MCT induced PH+KSF(scFv)-mIL9-KSF(scFv) (KSF-IL9) (n=6)

FIG. 7 shows representative right ventricular pressure (RVP) curves for 1 mouse per treatment group as recorded by invasive right heart catheterization. Column 2 shows the curves showing typical right ventricular morphology, column 3 provides the average cyclic maximum values (equates to systolic RVP=RVPsys) for each individual animal as calculated using LabChart software (in mmHg) and column 1 assigns the corresponding experimental group.

FIG. 8 shows the systolic right ventricular pressure (RVPsys) values (mean±standard deviation; in mmHg) in the 5 experimental groups. Compared to the sham induced controls, RVPsys was significantly elevated in all MCT induced PH groups (p<0.05), except the group treated with F8-IL9 (p=0.068). Compared to the MCT induced PH group without treatment, there was a significant reduction in RVPsys in the group treated with MACI (p=0.025) and the group treated with F8-IL9 (p=0.028). In the group treated with F8-IL9, RVPsys was significantly reduced compared to the group treated with KSF-IL9 (p=0.047).

FIGS. 9A-9B: shows basal (FIG. 9A) and medial (FIG. 9B) right ventricular diameter (RV diameter) values (mean±standard; in mm) in the 5 experimental groups. (FIG. 9A): Compared to the sham induced controls, the RV basal diameter was significantly increased in all MCT-induced PH groups, including the F8-IL9 treatment group (p<0.05). Compared to the MCT-induced PH group without treatment, there was a significant reduction in the RV basal diameter only in the group treated with F8-IL9 (p=0.014). (FIG. 9B): Compared to the sham induced controls, RV medial diameter was significantly increased in all MCT-induced PH groups (p<0.05), except the group treated with F8-IL9 (p=0.128). In the group treated with F8-IL9, both the RV basal diameter and RV medial diameters were significantly reduced compared to the group treated with KSF-IL9 (p=0.010 for RV basal diameter and p=0.037 for RV medial diameter).

FIG. 10 shows the right ventricular length (RV length) values (mean±standard; in mm) for the 5 experimental groups. Compared to the sham induced controls, the RV length was significantly increased in all MCT-induced PH groups (p<0.05), except the group treated with F8-IL9 (p=0.100). Compared to the MCT-induced PH group without treatment, there was a significantly decreased RV length only in the group treated with F8-IL9 (p=0.006). In the group treated with F8-IL9, RV length diameters were significantly reduced compared to the group treated with KSF-IL9 (p=0.016).

FIG. 11 shows the right ventricular fractional area change (RV FAC) values (mean±standard; in %) in the 5 experimental groups. Compared to sham induced controls, the RV FAC was significantly reduced in all MCT-induced PH groups (p<0.05), except the group treated with F8-IL9 (p=0.337). Compared to the MCT-induced PH group without treatment, there was a significant improvement in the RV FAC values in the group treated with MACI (p=0.032) and the group treated with F8-IL9 (p=0.046). There were no significant differences with respect to the RV FAC values when comparing the group treated with F8-IL9 and the group treated with KSF-IL9 (p=0.068).

FIGS. 12A-12C: shows the schematic structure of the F8 (IgG4-HC)-mIL9 and KSF (IgG4-HC)-mIL9 conjugates (FIG. 12A), and the results of Size Exclusion Chromatography analysis (left panel) and SDS-PAGE analysis under non reducing (nr) and reducing (r) conditions (right panel) of the F8 (IgG4-HC)-mIL9 conjugate (FIG. 12B), and of the KSF (IgG4-HC)-mIL9 conjugate (FIG. 12C).

FIG. 13 shows the systolic right ventricular pressure (RVPsys) values (mean±standard deviation; in mmHg) in the 7 experimental groups. Compared to sham induced controls, RVPsys was significantly elevated in all MCT induced PH groups (p<0.05) except for the group treated with F8_(IgG-HC4)-mIL9 (p=0.156). Compared to the MCT induced PH group without treatment, there was a significant attenuation in the group treated with MACI (p=0.004), the group treated with F8mIL9F8 in CrAb format (p=0.007) and the group treated with F8_(IgG-HC4)-mIL9 (p=0.007).

FIG. 14 shows the basal right ventricular diameter (RV diameter) values (mean±standard; in mm) in the 7 experimental groups. As compared to sham induced controls, RV basal diameter was significantly increased (p<0.05) in all MCT induced PH groups except for the groups treated with F8mIL9F8 in CrAb format (p=0.125) or F8_(IgG-HC4)-mIL9 (p=0.192). As compared to the MCT induced PH group without treatment, there was a significant attenuation in the group treated with F8mIL9F8 in CrAb format (p=0.019) and the group treated with F8_(IgG-HC4)-mIL9 (p=0.014).

FIG. 15 shows the tricuspid annular plane systolic excursion (TAPSE) values (mean±standard; in mm) in the 7 experimental groups. As compared to sham induced controls, TAPSE was significantly reduced (p<0.05) in all MCT induced PH groups except of the group treated with F8_(IgG-HC4)-mIL9 (p=0.175). As compared to the MCT induced PH group without treatment, there was a significant improvement of TAPSE in the groups treated with F8mIL9F8 in CrAb format (p=0.026) or F8_(IgG-HC4)-mIL9 (p=0.008) but not in any of the other treatment groups including the group treated with MACI (p=n.s.)

FIG. 16 shows the right atrial area (RA area) values (mean±standard; in mm) in the 7 experimental groups. Compared to sham induced controls, RA area was significantly increased (p<0.05) in all MCT induced PH groups except for the group treated with F8mIL9F8 in CrAb format (p=0.440) and the group treated with F8_(IgG-HC4)-mIL9 (p=0.537). Compared to the MCT induced PH group without treatment, there was a significant reduction in RA area in the groups treated with F8mIL9F8 in CrAb format (p=0.027) or F8_(IgG-HC4)-mIL9 (p=0.011) but not in any of the other treatment groups, including the group treated with MACI (p=n.s.)

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Conjugate

Conjugates as referred to herein comprise IL9, and one or more, e.g. one or two, specific binding members. The IL9 may be conjugated to N-terminus or C-terminus of the specific binding member.

Where the conjugate comprises two specific binding members, e.g. two scFvs, a first specific binding member may be conjugated at the N-terminus of IL9 and a second specific binding member may be conjugated at the C-terminus of IL9. The first and second specific binding members preferably have the same specificity.

Where the conjugate comprises two specific binding members, such as two scFvs, the N-terminus of the first specific binding member and the C-terminus of the second specific binding member are preferably free. “Free” in this context refers to the N- or C-terminus not being linked or otherwise conjugated to another moiety, such as IL9.

The conjugate in this context preferably comprises only one IL9.

The conjugate may be or may comprise a single-chain protein. When the conjugate is a single-chain protein, the entire protein can be expressed as a single polypeptide. For example, the conjugate may be a single-chain protein comprising IL9 and two single-chain Fvs (scFvs). The single-chain protein may be a fusion protein, for example a single-chain fusion protein comprising IL9 and two scFvs. By “single-chain fusion protein” is meant a polypeptide that is a translation product resulting from the fusion of two or more genes or nucleic acid coding sequences into one open reading frame (ORF). The fused expression products of the genes in the ORF may be conjugated by peptide linkers encoded in-frame. Suitable peptide linkers are described herein.

Where the conjugate comprises an immunoglobulin, in particular an IgG, such as an IgG4 molecule, the N-terminus of a first IL9 may be conjugated at the C-terminus of the first immunoglobulin heavy chain and the N-terminus of a second IL9 may be conjugated at the C-terminus of the second immunoglobulin heavy chain, for example via a peptide linker. The conjugate in this context comprises two IL9 moieties.

In an alternative embodiment, the conjugate may comprise an immunoglobulin, in particular an IgG, such as an IgG4 molecule, and a single IL9 moiety, wherein the N-terminus of the IL9 is conjugated at the C-terminus of one of the two immunoglobulin heavy chains, for example via a peptide linker.

Specific Binding Member

The specific binding member(s) in the conjugates described herein are preferably antibody molecules or antigen binding fragments thereof. In some preferred embodiments, the specific binding member comprises or consist of a single chain Fv (scFv), diabody, single-chain diabody, or an immunoglobulin (Ig) molecule, such as IgG. Most preferably, the specific binding member is an scFv or IgG molecule, such as IgG4.

In some embodiments, a first scFv may be conjugated at the N-terminus of human IL9 and a second scFv, with the same specificity as the first scFv, may be conjugated at the C-terminus of human IL9 (i.e. scFv-IL9-scFv). The resulting conjugate showed superior stability compared to a conjugate comprising IL9 conjugated to a single diabody.

ScFvs comprise the VH and VL domains of an antibody, wherein these domains are present in a single polypeptide chain. Preferably, the Fv polypeptide further comprises a polypeptide linker between the VH and VL domains which enables the ScFv to form the desired structure for antigen binding. For a review of scFv see Pluckthun in The Pharmacology of Monoclonal Antibodies, vol. 113, Rosenburg and Moore eds., Springer-Verlag, New York, pp. 269-3 15 (1994). The polypeptide linker between the VH and VL domains usually consists of at least 10 amino acids.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO1994/13804; Holliger and Winter, Cancer Immunol. Immunother. (1997) 45:128-130; Holliger et al., Proc. Natl. Acad. Sci. USA 90 6444-6448, 1993).

In a diabody (“db”) a heavy chain variable domain (VH) is connected to a light chain variable domain (VL) on the same polypeptide chain. The VH and VL domains are connected by a peptide linker that is too short to allow pairing between the two domains (generally around 5 amino acids). This forces paring with the complementary VH and VL domains of another chain. The VH and VL domains in a diabody are thus preferably linked by a 5 amino acid linker.

Alternatively, a diabody may be a single chain diabody (“ScDb”). In a single chain diabody two sets of VH and VL domains are connected together in sequence on the same polypeptide chain. For example, the two sets of VH and VL domains may be assembled in a single chain sequence as follows: (VH-VL)-(VH-VL), where the brackets indicate a set. In the single chain diabody format each of the VH and VL domains within a set is connected by a short or ‘non-flexible’ peptide linker. This type of peptide linker sequence is not long enough to allow pairing of the VH and VL domains within the set. Generally, a short or ‘non flexible’ peptide linker is around 5 amino acids. The two sets of VH and VL domains are connected as a single chain by a long or ‘flexible’ peptide linker. This type of peptide linker sequence is long enough to allow pairing of the VH and VL domains of the first set with the complementary VH and VL domains of the second set. Generally, a long or ‘flexible’ linker is around 15 amino acids. Single chain diabodies have been previously generated (Kontermann, R. E., and Muller, R. (1999), J. Immunol. Methods 226: 179-188). A bispecific single chain diabody has been used to target adenovirus to endothelial cells (Nettelbeck et al., Molecular Therapy (2001) 3, 882-891).

In a preferred embodiment, an immunoglobulin (Ig) molecule (also called antibody molecule) comprising two heavy chains and two light chains, may be conjugated to IL9 (i.e. IgG-IL9). In one embodiment, one or both, but preferably both, of the heavy chains is conjugated at its C-terminus to the N-terminus of an IL-9 moiety, optionally via a peptide linker.

An immunoglobulin molecule is composed of two light chains and two heavy chains that are disulfide-bonded. From the N- to C-terminus, each heavy chain comprises a variable region (VH), also called a variable heavy domain or a heavy chain variable domain, followed by three constant domains (CH1, CH2, and CH3), also called a heavy chain constant region. Similarly, from the N- to C-terminus, each light chain comprises a variable region (VL), also called a variable light domain or a light chain variable domain, followed by a light chain constant domain (CL), also called a light chain constant region. The heavy chain of an antibody molecule may be assigned to one of five types, called α (IgA), δ (IgD), ε (IgE), γ (IgG), or μ (IgM), some of which may be further divided into subtypes, e.g. γ₁(IgG₁), γ₂ (IgG₂), γ₃ (IgG₃), γ₄ (Iga₄), α₁(IgA₁) and α₂ (IgA₂). The light chain of an antibody molecule may be assigned to one of two types, called kappa (κ) and lambda (λ), based on the amino acid sequence of its constant domain. There are five major classes of immunoglobulins defined by the type of constant domain or constant region possessed by its heavy chain: IgA, IgD, IgE, IgG, and IgM, and several of these may be further divided into subclasses (isotypes), e.g., IgG₁, IgG₂, IgG₃, IgG₄, IgA₁, and IgA₂. The immunoglobulin heavy chain of an IgG molecule has the domain structure VH-CH1-CH2-CH3. The antibody light chain of an IgG antibody molecule has the domain structure VL-CL.

The specific binding members preferably bind an antigen associated with tissue and/or vascular remodelling, such as fibronectin. Preferably, the specific binding members may bind the Extra Domain-A (ED-A) of fibronectin.

Where the conjugate comprises more than one, e.g. two specific binding members, the specific binding members in the conjugates preferably have the same specificity (i.e. the conjugate is monospecific) and bind to the same antigen associated with tissue and/or vascular remodelling. For example, the conjugates may comprise two copies of the same specific binding member.

The specific binding member(s) may comprise an antigen binding site having the complementarity determining regions (CDRs), or the VH and/or VL domains of an antibody capable of binding to an antigen associated with tissue and/or vascular remodelling, such as the ED-A of fibronectin.

Thus, the specific binding member(s) may comprise an antigen binding site of antibody F8, which is known to bind ED-A. The specific binding member(s) may comprise an antigen binding site having one, two, three, four, five or six CDRs, or the VH and/or VL domains of antibody F8. The specific binding member(s) may comprise or consist of the sequence of antibody F8 in scFv format.

The specific binding member(s) may comprise or consist of the sequence of the F8 antibody molecule. For example, the F8 antibody molecule may be an IgG, IgA, IgE or IgM or any of the isotype sub-classes, particularly IgG1 or IgG4. IgG4 molecules exhibit reduced binding affinity to Fc receptors and reduced effector functions as compared to IgG1 molecules. In some embodiments, the IgG-class antibody molecule comprised in the conjugate of the invention is an IgG4-subclass antibody molecule, particularly a human IgG4-subclass antibody molecule. In one embodiment, the IgG4-subclass antibody molecule comprises an amino acid substitution in the Fc region at position S228, specifically the amino acid substitution S228P numbered according to the EU numbering system (also called the EU index), corresponding to the amino acid substitution S226P in the F8 heavy chain amino acid sequence set forth in SEQ ID NO: 60. Optionally, the F8 heavy chain set forth in SEQ ID NO: 60 may further comprise a C-terminal lysine.

The amino acid sequences of the CDRs of F8 are:
SEQ ID NO:1 (CDR1 VH);
SEQ ID NO:2 (CDR2 VH);
SEQ ID NO:3 (CDR3 VH);
SEQ ID NO:4 (CDR1 VL);
SEQ ID NO:5 (CDR2 VL), and/or
SEQ ID NO:6 (CDR3 VL).

The amino acid sequences of the VH and VL of F8 are:
SEQ ID NO: 7 (VH)
SEQ ID NO: 8 (VL)

The amino acid sequences of the IgG4 heavy and light chains of F8 are:
SEQ ID NO: 60 (heavy chain)
SEQ ID NO: 61 (light chain)

A specific binding member may comprise a VH domain having at least 70%, more preferably one of at least 75%, 80%, 85%, 90%, 95%, 96%, 97%, 98%, 99% or 100%, sequence identity to the F8 VH domain amino acid sequence of SEQ ID NO: 7.

A specific binding member may comprise a VL domain having at least 70%, more preferably one of at least 75%, 80%, 85%, 90%, 95%, 96%, 97%, 98%, 99% or 100%, sequence identity to the F8 VL domain amino acid sequence of SEQ ID NO: 8.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Altschul et al., Nucl. Acids Res. (1997) 25: 3389-3402) may be used.

Variants of these VH and VL domains and CDRs may also be employed in specific binding members for use in the conjugates described herein. Suitable variants can be obtained by means of methods of sequence alteration, or mutation, and screening. Particular variants for use as described herein may include one or more amino acid sequence alterations (addition, deletion, substitution and/or insertion of an amino acid residue), may be less than about 20 alterations, less than about 15 alterations, less than about 10 alterations or less than about 5 alterations, 4, 3, 2 or 1. Alterations may be made in one or more framework regions and/or one or more CDRs. In particular, alterations may be made in VH CDR1, VH CDR2 and/or VH CDR3.

A scFv for use in the invention may have at least 70%, more preferably one of at least 75%, 80%, 85%, 90%, 95%, 96%, 97%, 98%, 99% or 100%, sequence identity to the amino acid sequence of the F8 scFv set forth in SEQ ID NO: 9.

Preferably, the specific binding members comprise the CDRs, VH and/or VL domains, the sequence of the F8 scFv or the sequence of the F8 IgG.

ED-A of Fibronectin

The alternatively spliced ED-A domain of fibronectin (ED-A) is a 90 amino acid sequence which is inserted into the extracellular matrix (ECM) component fibronectin (FN) through alternative splicing and is located between domain 11 and 12 of FN (Borsi et al. (1987), J. Cell. Biol.). The ED-As of mouse fibronectin and human fibronectin are 96.7% identical (only 3 amino acids differ between the two 90 amino acid sequences).

Expression of ED-A in the healthy adult is confined to vascular structures in few tissues in which physiological angiogenesis takes place, namely the placenta, the endometrium in the proliferative phase and some vessels in the ovaries (Schwager et al. (2009) Arthritis Res. Ther.). ED-A is also abundant during tissue remodeling, fibrosis (such as liver and pulmonary fibrosis), and in vascular tissue and stroma of many cancer types. Furthermore, the expression of ED-A in a MCT-induced model of pulmonary hypertension has been reported in Franz et al., Oncotarget, 2016, 7, 81241-81254.

Over the years, the current applicant has developed a number of anti-cancer agents, including targeted cytokines (“immunocytokines”) based on the anti-ED-A antibody “F8”.

Reference to the work on the anti-ED-A “F8” antibody and conjugates thereof can be found in WO2008/120101, WO2009/013619, WO2009/056268, WO2010/078945, WO2010/078950, WO2011/015333, WO2012/041451, WO2013/014149, WO2014/055073, WO2014/173570, WO2014/174105, WO2015/114166, WO2016/180715, WO2017/009469, WO2018/069467, WO2018/087172, WO2018/224550, WO2019/185792, and WO2020/070150.

F8 expressed in diabody format and conjugated to two IL9 moieties has been reported in Veneto et al., PNAS, 2015: 112(7):2000-2005.

Interleukin 9 The conjugate comprises IL9. The IL9 may be mammalian IL9, preferably human IL9. The amino acid sequence of human IL9 is set out in SEQ ID NO: 16. The conjugate of the invention preferably comprises a single IL9 polypeptide.

Typically, the IL9 has at least 70%, more preferably one of at least 75%, 80%, 85%, 90%, 95%, 96%, 97%, 98%, 99% or 100%, sequence identity to the amino acid sequence shown in SEQ ID NO: 16.

IL9 in conjugates of the invention retains a biological activity of IL9, e.g. the ability to activate T cells, eosinophils, type 2 innate lymphoid cells (ILC2), and/or mast cells. In particular, retention of biological activity of IL9 in conjugates of the invention may be tested by determining the ability of the conjugate to stimulate proliferation of mast cells.

Where the conjugate comprises a single specific binding member, the IL9 may be conjugated to the N- or C-terminus of the specific binding member.

Where the conjugate comprises two specific binding members, e.g. two scFvs, the N-terminus of IL9 is preferably conjugated to a first specific binding member and the C-terminus of IL9 is conjugated to a second specific binding member.

IL9 is known to comprise four glycosylation sites and glycosylation of IL9 has been reported to determine extravasion and tumour-targeting properties of IL-9 containing Immunoconjugates (Venetz et al. PNAS, 2015, 112(7):2000-2005). In one embodiment, the IL9 may be a non-glycosylated form of IL9. For example, the human IL9 may be a variant of human IL9 in which one or more of the four glycosylation sites have been removed by substituting one or more of the asparagine (N) residues at positions 32, 45, 60 and/or 96 of the sequence of human IL9 as set forth in SEQ ID NO: 16 with alanine (A) or glutamine (Q). Alternatively, non-glycosylated forms of IL9 may be prepared by glycan engineering or enzymatic de-glycosylation. The non-glycosylated form or variant of IL9 preferably retains one or more biological activities of IL9 as described herein.

Linkers

In the conjugate, the specific binding member, e.g. scFv, and IL9 may be connected to each other directly, for example through any suitable chemical bond, but preferably are connected by a peptide linker. The peptide linker may be a short (2-30, preferably 10-20) residue stretch of amino acids. Suitable examples of peptide linker sequences are known in the art. One or more different linkers may be used.

The linker connecting the specific binding member and IL9 in the conjugate may be 15 amino acids in length. An example of a suitable linker sequence is set forth in SEQ ID NO: 27.

In a preferred embodiment, the specific binding member is an scFv and the linker connecting the scFv and IL9 in the conjugate is 10 amino acids in length. An example of a suitable linker sequence is set forth in SEQ ID NO: 28.

In an alternative preferred embodiment, the specific binding member is an IgG molecule and the linker connecting the heavy chain(s) of the IgG molecule and IL9 in the conjugate is 15 amino acids in length. An example of a suitable linker sequence is set forth in SEQ ID NO: 27.

ScFv antibody fragments comprise the VH and VL domains of an antibody, wherein these domains are present in a single polypeptide chain. Preferably, the Fv polypeptide further comprises a polypeptide linker between the VH and VL domains which enables the ScFv to form the desired structure for antigen binding. For a review of ScFv see Pluckthun in The Pharmacology of Monoclonal Antibodies, vol. 113, Rosenburg and Moore eds., Springer-Verlag, New York, pp. 269-3 15 (1994). The polypeptide linker between the VH and VL domains usually consists of at least 10 amino acids.

Where the antibody molecule is an scFv, the VH and VL domains of the antibody are preferably linked by a 10 to 20 amino acid linker. For example, the VH and VL domains may be linked by an amino acid linker which is 10, 11, 12, 13, 14, 15, 16, 17, 18, 19, or 20 amino acid in length.

Suitable linker sequences are known in the art and include the linker sequences set forth in SEQ ID NO: 25.

The chemical bond may be, for example, a covalent or ionic bond. Examples of covalent bonds include peptide bonds (amide bonds) and disulphide bonds. For example, the specific binding member and IL9 may be covalently linked. For example, by peptide bonds (amide bonds).

Pulmonary Hypertension

Pulmonary hypertension (PH) refers to high blood pressure in the blood vessels that supply blood to the lungs (pulmonary arteries). PH is defined as a mean pulmonary arterial pressure (PAPm) of 25 mmHg at rest, measured by right heart catheterization (RHC). The normal PAPm at rest in healthy individuals is 14±3 mmHg with an upper limit of normal of approximately 20 mmHg (Galiè et al., ESC/ERS Guidelines for the diagnosis and treatment of pulmonary hypertension, European Respiratory Journal, 2015; 46: 903-975).

PH may be caused by heart or lung condition, associated with other medical conditions, such as connective tissue disorders or blood clots, or occur for unknown reasons. PH can be categorized into five groups according to their similar clinical presentation, pathological findings, haemodynamic characteristics and treatment strategy (Galiè et al., ESC/ERS Guidelines for the diagnosis and treatment of pulmonary hypertension, European Respiratory Journal, 2015; 46: 903-975):
Group 1: pulmonary arterial hypertension (PAH)
Group 2: PH due to left heart disease (LHD)
Group 3: PH due to lung disease and/or hypoxaemia
Group 4: Chronic thromboembolic pulmonary hypertension
Group 5: PH with unclear and/or multifactorial mechanisms

The mouse model of PH employed by the present inventors is thought to mimic not only PAH (Group 1) but also other groups of PH, insofar as they are in advanced stages and show pulmonary vascular remodelling as measured by elevated resistance in right heart catheterization (precapillary PH). Pulmonary vascular remodelling is the key structural alteration in PH and involves changes in intima, media, and adventitia of blood vessels, often with the interplay of inflammatory cells. Given the efficacy of a conjugate comprising IL9 and a specific binding member that binds ED-A in reducing pulmonary arterial pressure in said mouse model (Example 3), it is expected that the conjugates described herein are suitable for treating PH regardless of cause and independent of the PH Group from which the patient is suffering. This is particularly advantageous as few treatments for PH are available, with the exception of PAH (Group 1) which, however, is a rare form of PH. Furthermore, even in the case of PAH (Group 1) there remains a need in the art for treatments which can prevent the development of right heart failure. As ED-A is also expressed in the remodelled right ventricle in PH patients, the conjugates described herein are also expected to have a beneficial effect on the myocardium in PH patients. This is consistent with the results of echocardiographic assessment of surrogate parameters of right ventricular (RV) morphology and function (RV basal and medial diameters, RV length, and RV fractional area change in mice treated with a conjugate comprising IL9 and a specific binding member that binds ED-A (Example 3.2; FIGS. 9A, 9B, 10, and 11)

PH, as referred to herein, may thus be any type of PH, such as Group 1 PH, Group 2 PH, Group 3 PH, Group 4 PH, or Group 5 PH. In particular embodiments, the PH may be associated with pulmonary vascular remodelling and/or right ventricle remodelling.

Methods of Treatment

A conjugate according to the invention may be used in a method of treatment of the human or animal body, such as a method of treatment (which may include prophylactic treatment and/or curative treatment) of a pulmonary hypertension in a patient (typically a human patient) comprising administering the conjugate to the patient.

Accordingly, such aspects of the invention provide methods of treatment comprising administering a conjugate of the invention, or pharmaceutical compositions comprising such a conjugate, for the treatment of pulmonary hypertension in a patient, and a method of making a medicament or pharmaceutical composition comprising formulating the conjugate of the present invention with a physiologically acceptable carrier or excipient.

Thus, a conjugate of the invention may be for use in a method of treating pulmonary hypertension. Also contemplated is a method of treating pulmonary hypertension in a patient, the method comprising administering a therapeutically effective amount of a conjugate of the invention to the patient. Also provided is the use of a conjugate of the invention for the manufacture of a medicament for the treatment of pulmonary hypertension. Further provided is a conjugate of the invention for use in a method of delivering IL9 to sites remodelled lung and/or heart tissue or vasculature in a patient with pulmonary hypertension, as well as a method of delivering IL9 to sites of remodelled lung and/or heart tissue or vasculature in a patient with pulmonary hypertension comprising administering to the patient a conjugate of the invention.

Types of pulmonary hypertension treatable using the conjugate of the invention include Group 1, Group 2, Group 3, Group 4 and Group 5 pulmonary hypertension. Treatment may include prophylactic treatment.

Pharmaceutical Compositions

The conjugate may be in the form of a pharmaceutical composition comprising at least one conjugate and optionally a pharmaceutically acceptable excipient.

Pharmaceutical compositions typically comprise a therapeutically effective amount of a conjugate and optionally auxiliary substances such as pharmaceutically acceptable excipient(s). Said pharmaceutical compositions are prepared in a manner well known in the pharmaceutical art. A carrier or excipient may be a liquid material which can serve as a vehicle or medium for the active ingredient. Suitable carriers or excipients are well known in the art and include, for example, stabilisers, antioxidants, pH-regulating substances, controlled-release excipients. The pharmaceutical preparation of the invention may be adapted, for example, for parenteral use and may be administered to the patient in the form of solutions or the like.

Compositions comprising the conjugate may be administered to a patient. Administration is preferably in a “therapeutically effective amount”, this being sufficient to show benefit to the patient. Such benefit may be at least amelioration of at least one symptom. The actual amount administered, and rate and time-course of administration, will depend on the nature and severity of what is being treated. Prescription of treatment, e.g. decisions on dosage etc., is within the responsibility of general practitioners and other medical doctors. Treatments may be repeated at daily, twice-weekly, weekly, or monthly intervals at the discretion of the physician.

Conjugates may be administered to a patient in need of treatment via any suitable route, usually by injection into the bloodstream and/or directly into the site to be treated. The precise dose and its frequency of administration will depend upon a number of factors, such as the route of treatment.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A pharmaceutical composition comprising a conjugate may be administered alone or in combination with other treatments, either simultaneously or sequentially dependent upon the type of pulmonary hypertension to be treated.

Kits

Another aspect of the invention provides a therapeutic kit for use in the treatment of pulmonary hypertension comprising a conjugate of the invention. The components of a kit are preferably sterile and in sealed vials or other containers. A kit may further comprise instructions for use of the components in a method of the invention. The components of the kit may be comprised or packaged in a container, for example a bag, box, jar, tin or blister pack.

Nucleic Acids, Vectors, Host Cells and Methods of Production

Provided is an isolated nucleic acid molecule encoding a conjugate according to the invention. Nucleic acid molecules may comprise DNA and/or RNA and may be partially or wholly synthetic.

Further provided are constructs in the form of plasmids, vectors, transcription or expression cassettes which comprise such nucleic acids. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids e.g. phagemid, or viral e.g. ‘phage, as appropriate. For further details, see, for example, Sambrook & Russell (2001) Molecular Cloning: a Laboratory Manual: 3rd edition, Cold Spring Harbor Laboratory Press. Many known techniques and protocols for manipulation of nucleic acid, for example in the preparation of nucleic acid constructs, mutagenesis, sequencing, introduction of DNA into cells and gene expression, and analysis of proteins, are described in detail in Ausubel et al. (1999) 4th eds., Short Protocols in Molecular Biology: A Compendium of Methods from Current Protocols in Molecular Biology, John Wiley & Sons.

A recombinant host cell that comprises one or more constructs as described above is also provided. Suitable host cells include bacteria, mammalian cells, plant cells, filamentous fungi, yeast and baculovirus systems and transgenic plants and animals.

A conjugate according to the present invention may be produced using such a recombinant host cell. The production method may comprise expressing a nucleic acid or construct as described above. Expression may conveniently be achieved by culturing the recombinant host cell under appropriate conditions for production of the conjugate. Following production, the conjugate may be isolated and/or purified using any suitable technique, and then used as appropriate. The conjugate may be formulated into a composition including at least one additional component, such as a pharmaceutically acceptable excipient.

Systems for cloning and expression of a polypeptide in a variety of different host cells are well known. The expression of antibodies, including conjugates thereof, in prokaryotic cells is well established in the art. For a review, see for example Plückthun (1991), Bio/Technology 9: 545-551. A common bacterial host is E. coli.

Expression in eukaryotic cells in culture is also available to those skilled in the art as an option for production of conjugates for example Chadd et al. (2001), Current Opinion in Biotechnology 12: 188-194); Andersen et al. (2002) Current Opinion in Biotechnology 13: 117; Larrick & Thomas (2001) Current Opinion in Biotechnology 12:411-418. Mammalian cell lines available in the art for expression of a heterologous polypeptide include Chinese hamster ovary (CHO) cells, HeLa cells, baby hamster kidney cells, NS0 mouse melanoma cells, YB2/0 rat myeloma cells, human embryonic kidney cells, human embryonic retina cells and many others.

A method comprising introducing a nucleic acid or construct disclosed herein into a host cell is also described. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques may include calcium chloride transformation, electroporation and transfection using bacteriophage.

The nucleic acid or construct may be integrated into the genome (e.g. chromosome) of the host cell. Integration may be promoted by inclusion of sequences that promote recombination with the genome, in accordance with standard techniques.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—Preparation of IL9 Conjugates

1.1 Cloning of IL9-Containing Conjugates

The genes encoding conjugates (fusion proteins) comprising murine IL9 (mIL9) or human IL9 (hIL9) were generated using PCR assembly. For all conjugates, a sequence encoding an IgG-derived signal peptide (SP) was added at the N-terminus of the conjugates to enable secretion of the encoded conjugates. Using engineered HindIII and NotI restriction sites, the genes were cloned into a suitable mammalian expression vector. The signal peptides were cleaved after expression of the conjugates and thus were not part of the mature conjugates.

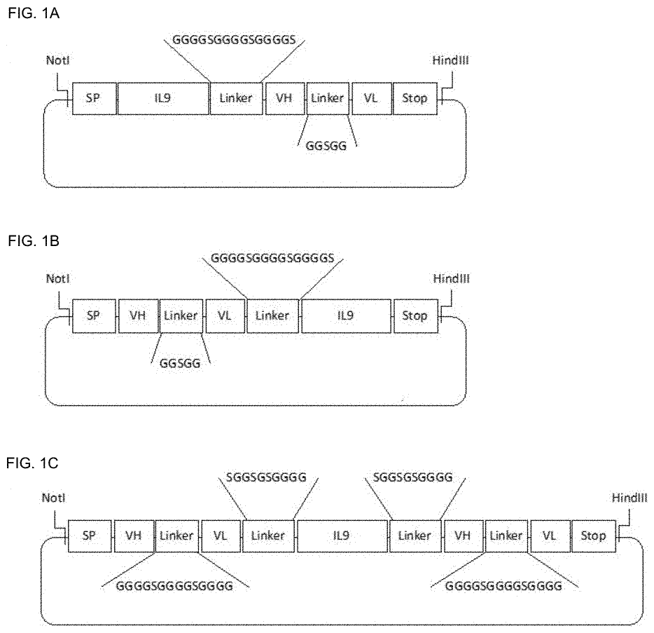

A schematic illustration of the gene assembly and corresponding expression plasmid for the various conjugates is shown in FIGS. 1A-1C.

1.1.1. Cloning of F8-IL9 Conjugates

1.1.1.1 Cloning of the F8(scFv)-mIL9-F8(scFv) and F8(scFv)-hIL9-F8(scFv) Conjugates

For F8(scFv)-mIL9-F8(scFv) and F8(scFv)-hIL9-F8(scFv), IL9 (lacking the signal peptide sequence) was conjugated at its N- and C-terminus to an F8 scFv via a 10 amino acid linker (SGGSGSGGGG) to produce a conjugate in “CrAb” format.

To clone the F8(scFv)-IL9-F8(scFv) conjugates, a synthetic gene encoding hIL9 or mIL9 containing an EcoRI silent mutation and a 10 amino acid linker at the 5′ and the 3′ ends and F8-ScFv in pcDNA3.1 were used as templates. Fragment “Leader_sequence-ScFvF8-linker” was amplified from F8-ScFv using the following primers in accordance to the desired PCR product:

Leader_sequence-ScFvF8-linker

| Payload | Forward Primer | Backward Primer |
|---|---|---|
| hIL9 and | TCCTCCTGTTCCTCGTCGCTGTGGCTACAG | ACCTCCACCGCCAGAACCACTTCCGCCTGA |
| mIL9 | GTGTGCACTCGGAGGTGCAGCTGTTGGAGT | TTTGATTTCCACCTTGGTCCCTTG - SEQ |
| | CTGGG - SEQ ID NO: 29 | ID NO: 30 |

The different “Linker-IL9-Linker” were amplified from Linker-IL9-Linker gene using the following primers in accordance with the desired PCR product:

Linker-IL9-Linker

| Payload | Forward Primer | Backward Primer |
|---|---|---|
| hIL9 | tcaggcggaagtggttctggcggtggaggt | cccctgaaccactgcctccagaTATCTTGC |
| | CAGGGGTGTCCAACCTTGGCGGGG - SEQ | CT - SEQ ID NO: 32 |
| | ID NO: 31 | |
| mIL9 | tcaggcggaagtggttctggcggtggaggt | ccccctgaaccactgcctccagaTGGTCGG |
| | CAGAGATGCAGCACCACATGGGG - SEQ | CTT - SEQ ID NO: 34 |
| | ID NO: 33 | |

The different “Linker-ScFv8-STOP-NotI” were amplified from F8-ScFv using the following primers in accordance with the desired PCR product:

Linker-ScFv8-STOP-Notl

| Payload | Forward Primer | Backward Primer |
|---|---|---|
| hIL9 and | gcagtggttcagggggcggtggaGAGGTGC | TTTTCCTTTTGCGGCCGCTTATTTGATTTC |
| mIL9 | AGCTGT - SEQ ID NO: 35 | CACCTTGGTCCC - SEQ ID NO: 36 |

The different “HindIII-Leader_sequence-ScFvF8-Linker-IL9-Linker” were amplified from PCRs “Leader_sequence-ScFvF8-linker” and “Linker-IL9-Linker” using the following primers in accordance with the desired PCR product:

HindIII-Leader_sequence-ScFvF8-Linker-IL9-Linker

| Payload | Forward Primer | Backward Primer |
|---|---|---|
| hIL9 | CCCAAGCTTGTCGACCATGG | cccctgaaccactgcctcca |
| | GCTGGAGCCTGATCCTCCTG | gaTATCTTGCCT - SEQ |
| | TTCCTCGTCGCTGTGGC - | ID NO: 32 |
| | SEQ ID NO: 37 | |
| mIL9 | CCCAAGCTTGTCGACCATGG | ccccctgaaccactgcctcc |
| | GCTGGAGCCTGATCCTCCTG | agaTGGTCGGCTT - SEQ |
| | TTCCTCGTCGCTGTGGC - | ID NO: 34 |
| | SEQ ID NO: 37 | |

The different “Linker-IL9-Linker-ScFv8-STOP-NotI” were amplified from PCRs “Linker-IL9-Linker” and “Linker-ScFv8-STOP-NotI” using the following primers according to the desired PCR product:

Linker-IL9-Linker-ScFv8-STOP-NotI

| Payload | Forward Primer | Backward Primer |
|---|---|---|
| hIL9 | CAGGGGTGTCCAAC | TTTTCCTTTTGCGGCCGCTTAT |
| | CTTGGCGGGGA - | TTGATTTCCACCTTGGTCCC - |
| | SEQ ID NO: 38 | SEQ ID NO: 36 |
| mIL9 | CAGAGATGCAGCAC | TTTTCCTTTTGCGGCCGCTTAT |
| | CACATGGGGA - | TTGATTTCCACCTTGGTCCC - |
| | SEQID NO: 39 | SEQ ID NO: 36 |

Fragment HindIII-Leader_sequence-ScFvF8-Linker-IL9-Linker was digested by HindIII and EcoRI and ligated into the pcDNA3.1 plasmid. Fragment Linker-IL9-Linker-ScFv8-STOP-NotI was digested by EcoRI and NotI and ligated into the pcDNA3.1 plasmid containing HindIII-Leader_sequence-ScFvF8-Linker-IL9.

The amino acid sequence of the F8(scFv)-mIL9-F8(scFv) and F8(scFv)-hIL9-F8(scFv) conjugates are shown in SEQ ID NOs: 17 and 18, respectively.

1.1.1.2 Cloning of the F8(db)-mIL9 and F8(db)-hIL9 Conjugates

With the aid of a 15 amino acid linker (GGGGSGGGGSGGGGS), the diabody (db) version of the F8 antibody was conjugated to murine or human IL9 at the C-terminus of the db, to prepare conjugates F8(db)-mIL9 and F8(db)-hIL9.

To clone conjugates F8(db)-mIL9 and F8(db)-hIL9, the human or murine version of F8(scFv)-IL9-F8(scFv) in pcDNA3.1 and DbF8X in pcDNA3.1 were used as templates for the PCR. The fragment “HindIII-Leader_sequence-DbF8” was amplified from DbF8X using the following primers:

HindIII-Leader_sequence -DbF8

| Payload | Forward Primer | Backward Primer |
|---|---|---|
| hIL9 | ATTAAAGCTTCCACCATGG | GCCAGAGCCACCTCCGCCTG |
| and | GCTGGAGCCTGATCCTCCT | AACCGCCTCCACCTTTGATT |
| mIL9 | GTTCCTCGTCGCTGTGG | TCCACCTTGGTCCC - |
| | C - SEQ ID NO: 40 | SEQ ID NO: 41 |

Fragments “IL9-Stop-NotI” were amplified from F8(scFv)-mIL9-F8(scFv) or F8(scFv)-hIL9-F8(scFv) using the following primers in accordance with the desired PCR product:

IL9-Stop-NotI

| Payload | Forward Primer | Backward Primer |
|---|---|---|
| hIL9 | CAGGCGGAGGTGGCTCTGGC | TAATGCGGCCGCTTATCAT |
| | GGTGGCGGATCACAGGGGTG | ATCTTGCCTCTCATCCCTC |
| | TCCAACCTTG - SEQ ID | T - SEQ ID NO: 43 |
| | NO: 42 | |
| mIL9 | CAGGCGGAGGTGGCTCTGGC | TAATGCGGCCGCTTATCAT |
| | GGTGGCGGATCACAGAGATG | GGTCGGCTTTTCTGCCTTT |
| | CAGCACCACA - SEQ ID | GCAT - SEQ ID NO: 45 |
| | NO: 44 | |

Fragments “HindIII-Leader_sequence-DbF8” and “IL9-Stop-NotI” were assembled by means of PCR and double digested with HindIII and NotI-HF restriction enzymes. The digested DNA fragments were ligated into the pcDNA3.1 plasmid.

The amino acid sequences of the F8(db)-mIL9 and F8(db)-hIL9 conjugates are shown in SEQ ID NOs: 20 and 22.

1.1.1.3 Cloning of mIL9-F8(db) and hIL9-F8(db) Conjugates

With the aid of a 15 amino acid linker (GGGGSGGGGSGGGGS), the diabody (db) version of F8 antibody was conjugated to murine or human IL9 at the N-terminus of the db, to prepare conjugates mIL9-F8(db) and hIL9-F8(db).

To clone conjugates mIL9-F8(db) and hIL9-F8(db), human or murine version of F8(scFv)-IL9-F8(scFv) in pcDNA3.1 and DbF8X in pcDNA3.1 were used as templates for the PCR. Fragments “Leader_sequence-IL9” were amplified from F8(scFv)-mIL9-F8(scFv) or F8(scFv)-hIL9-F8(scFv) using the following primers:

Leader_sequence-IL9

| Payload | Forward Primer | Backward Primer |
|---|---|---|
| hIL9 | TCCTCCTGTTCCTCGTCGCTG | GCCAGAGCCACCTCCGCCTG |
| | TGGCTACAGGTGTGCACTCGC | AACCGCCTCCACCTATCTTG |
| | AGGGGTGTCCAACCTTG - | CCTCTCATCCCTCT - SEQ |
| | SEQ ID NO: 46 | ID NO: 47 |
| mIL9 | TCCTCCTGTTCCTCGTCGCTG | GCCAGAGCCACCTCCGCCTG |
| | TGGCTACAGGTGTGCACTCGC | AACCGCCTCCACCTGGTCGG |
| | AGAGATGCAGCACCACA - | CTTTTCTGCCTTTGCAT - |
| | SEQ ID NO: 48 | SEQ ID NO: 49 |

Fragments “HindIII-Leader_sequence-IL9” were amplified from the fragment “Leader_sequence—DbF8” using the following primers:

HindIII-Leader_sequence -IL9

| Payload | Forward Primer | Backward Primer |
|---|---|---|
| hIL9 | ATTAAAGCTTCCACCATGGG | GCCAGAGCCACCTCCGCCT |
| | CTGGAGCCTGATCCTCCTGT | GAACCGCCTCCACCTATCT |
| | TCCTCGTCGCTGTGGC - | TGCCTCTCATCCCTCT - |
| | SEQ ID NO: 40 | SEQ ID NO: 47 |
| mIL9 | ATTAAAGCTTCCACCATGGG | GCCAGAGCCACCTCCGCCTG |
| | CTGGAGCCTGATCCTCCTGT | AACCGCCTCCACCTGGTCGG |
| | TCCTCGTCGCTGTGGC - | CTTTTCTGCCTTTGCAT - |
| | SEQ ID NO: 40 | SEQ ID NO: 49 |

The fragment “DbF8-Stop-NotI” was amplified from DbF8X using the following primers:

DbF8-Stop-NotI

| Payload | Forward Primer | Backward Primer |
|---|---|---|
| hIL9 | CAGGCGGAGGTGGCTCTGG | TAATGCGGCCGCTTATCATTT |
| and | CGGTGGCGGATCAGAGGTG | GATTTCCACCTTGGTCCC - |
| mIL9 | CAGCTGTTGGAGTCT - | SEQ ID NO: 51 |
| | SEQ ID NO: 50 | |

Fragments “HindIII-Leader_sequence-IL9” and “DbF8-Stop-NotI” were assembled by means of PCR and double digested with HindIII and NotI-HF restriction enzymes. The digested DNA fragments were ligated into the pcDNA3.1 plasmid.

The amino acid sequence of the mIL9-F8(db) and hIL9-F8(db) conjugates are shown in SEQ ID NOs: 21 and 23.

1.1.1.4 Cloning of the F8(IgG4-HC)-mIL9 Conjugate

The F8 (IgG4-HC)-mIL9 protein was expressed from the mammalian expression vector pMM137-F8IgG4-HC-mIL9. For the construction of the pMM137-F8IgG4-HC-mIL9 plasmid, a sequence encoding for a (G4S)3 peptidic linker and the murine IL9 sequence (SEQ ID NO: 15) was fused to the 3′-terminal portion of the F8 heavy chain sequence by a PCR assembly approach. Briefly, a fragment (termed Fragment-A), consisting of the CH1-Hinge-CH2-CH3 portions of the human IgG4 sequence was amplified by PCR using the pMM137-F8IgG4-5226P plasmid as template and the following primer pairs:

Fragment-A

| Payload | Forward Primer | Backward Primer |
|---|---|---|
| mIL9 | TTTGACTACTGGGGCCAGG | CCACCGCCAGAGCCACCTC |
| | GAACCCTGGTCACCGTCTC | CGCCTGAACCGCCTCCACC |
| | SEQ ID NO: 58 | ACCCAGAGACAGGGAGAGG |
| | | CTCTTC SEQ ID NO: 59 |

In parallel a fragment (termed Fragment-B) consisting of a 15 amino acid linker and mIL9 was amplified by PCR using as template a custom synthetized cDNA fragment and the following primer pairs:

Fragment-B

| Payload | Forward Primer | Backward Primer |
|---|---|---|
| mIL9 | CAGGCGGAGGTGGCTCTGG | TAATGCGGCCGCTTATCAT |
| | CGGTGGCGGATCACAGAGA | GGTCGGCTTTTCTGCCTTT |
| | TGCAGCACCACA SEQ ID | GCAT SEQ ID NO: 45 |
| | NO: 44 | |

Fragment-A and Fragment-B were then fused by PCR using the following primer pairs:

Fragment A and Fragment-B

| Payload | Forward Primer | Backward Primer |
|---|---|---|
| mIL9 | TTTGACTACTGGGGCCAG | TAATGCGGCCGCTTATCAT |
| | GGAACCCTGGTCACCGTC | GGTCGGCTTTTCTGCCTTT |
| | TC SEQ ID NO: 58 | GCAT SEQ ID NO: 45 |

The obtained amplicon (termed fragment A/B) was restriction digested with XhoI and NotI and inserted into the corresponding sites of the pMM137-F8IgG4-S226P plasmid giving rise to the pMM137-F8IgG4-HCmIL9 expression vector.

The amino acid sequence of the F8(IgG4-HC)-mIL9 conjugate is shown in SEQ ID NOs: 61 and 62.

1.1.2 Cloning of KSF-IL9 Conjugates

Conjugates were prepared using the same cloning strategy described above but replacing the F8 scFv or db with an anti-hen lysozyme KSF scFv or db (Frey et al. Integr. Biol., 2011, 3, 468-478).

1.1.2.1 Cloning of the KSF(scFv)-mIL9-KSF(scFv) conjugate:

For the cloning of KSF(scFv)-mIL9-KSF(scFv), the murine version of F8(scFv)-IL9-F8(scFv) in pcDNA3.1 and DbKSF-GMCSF in pcDNA3.1 were used as templates for PCR. The fragments “HindIII-Leader_sequence-KSF(VH)” and “KSF(VL)-linker” were amplified from DbKSF-GMCSF and then PCR assembled using the following primers:

| | | |
|---|---|---|
| Payload | Forward Primer | Backward Primer |

HindIII-Leader_sequence -KSF(VH)

| | | |
|---|---|---|
| mIL9 | ATTAAAGCTTCCACCATGGG | ACCGCCAGAGCCACCTCCGC |
| | CTGGAGCCTGATCCTCCTGT | CTGAACCGCCTCCACCACTC |
| | TCCTCGTCGCTGTGGC - | GAGACGGTGACCAGGG - |
| | SEQ ID NO: 40 | SEQ ID NO: 52 |

KSF(VL)-linker

| | | |
|---|---|---|
| mIL9 | AGGCGGTTCAGGCGGAGGTG | CTCTGacctccaccgccaga |
| | GCTCTGGCGGTGGCGGATCG | accacttccgcctgaGCCTA |
| | TCTGAGCTGACTCAGGA - | GGACGGTCAGCTTGGT - |
| | SEQ ID NO: 53 | SEQ ID NO: 54 |

A PCR containing “linker-mIL9-KSF(ScFv)-NotI” was amplified from a synthetic gene using the following primers:

linker-mIL9-KSF(ScFv)-NotI

| Payload | Forward Primer | Backward Primer |
|---|---|---|
| mIL9 | ATGGGGAATTCGAGACACC | taatGCGGCCGCTTATCAT |
| | AATTACCTTATTGAA - | CCCAGCACTGTCAGCTT - |
| | SEQ ID NO: 55 | SEQ ID NO: 56 |

“Linker-mIL9-KSF(ScFv)-NotI” was double digested with EcoRI and NotI and inserted into pcDNA3.1. The resulting plasmid was digested with HindIII and EcoRI for insertion of the assembly of HindIII-Leader_sequence-KSF, resulting in the full encoded gene inserted into pcDNA3.1.

The amino acid sequence of the KSF(scFv)-mIL9-KSF(scFv) conjugate is shown in SEQ ID NO: 19.

1.1.2.2 Cloning of the KSF(db)-mIL9 Conjugate:

To clone the KSF(db)-mIL9 conjugate, the murine version of F8(scFv)-IL9-F8(scFv) in pcDNA3.1 and DbKSF-GMCSF in pcDNA3.1 were used as templates for PCR. The fragment “HindIII-Leader_sequence-DbKSF” was amplified from DbF8X using the following primers:

HindIII-Leader_sequence -DbKSF

| Payload | Forward Primer | Backward Primer |
|---|---|---|
| mIL9 | ATTAAAGCTTCCACCATGGG | AATCGGCTGGCTGTGGTA |
| | CTGGAGCCTGATCCTCCTGT | TTCGGCGGAGGGACCAAG |
| | TCCTCGTCGCTGTGGC - | CTGACCGTCCTAGGCGGT |
| | SEQ ID NO: 40 | GGAGGCGGTTCAGGCGGA |
| | | GGTGGCTCTGGC - SEQ |
| | | ID NO: 57 |

Fragments “IL9-Stop-NotI” were amplified from F8(scFv)-mIL9-F8(scFv) using the following primers in accordance to the desired PCR product:

IL9-Stop-NotI

| Payload | Forward Primer | Backward Primer |
|---|---|---|
| mIL9 | CAGGCGGAGGTGGCTCTGG | TAATGCGGCCGCTTATCAT |
| | CGGTGGCGGATCACAGAGA | GGTCGGCTTTTCTGCCTTT |
| | TGCAGCACCACA - SEQ | GCAT - SEQ ID NO: 45 |
| | ID NO: 44 | |

Fragments “HindIII-Leader_sequence-DbKSF” and “IL9-Stop-NotI” were assembled by means of PCR and double digested with HindIII and NotI-HF restriction enzymes. The digested DNA fragments were ligated into the pcDNA3.1 plasmid.

The amino acid sequence of the KSF(db)-mIL9 conjugate is shown in SEQ ID NO: 24.

1.1.2.3 Cloning of the KSF(IgG4-HC)-mIL9 Conjugate

The KSF(IgG4-HC)-mIL9 protein was expressed from the mammalian expression vector pMM137-KSFIgG4-HC-mIL9. The pMM137-KSFIgG4-HC-mIL9 plasmid was constructed by inserting the XhoI-NotI fragment from the vector pMM137-F8IgG4-HCmIL9, into the corresponding sites of the vector pMM137-KSFIgG1.

The amino acid sequence of the KSF(IgG4-HC)-mIL9 conjugate is shown in SEQ ID NOs: 64 and 65.

1.2 Expression and Purification of IL9-Containing Conjugates

The IL9 conjugates (F8(scFv)-mIL9-F8(scFv), KSF(scFv)-mIL9-KSF(scFv), F8(scFv)-hIL9-F8(scFv), F8(db)-mIL9, KSF(db)-mIL9, F8(db)-hIL9, and hIL9-F8(db)) were expressed using transient gene expression in CHO cells. For 1 ml of production, 4×10⁶ cells were collected by centrifugation and resuspended in 1 mL of medium supplemented with 4 mM ultraglutamine. 0.625 μg of plasmid DNAs followed by 2.5 μg polyethylene imine (PEI; 1 mg/mL solution in water at pH 7.0) per million cells, were added to the cells and gently mixed. The transfected cultures were incubated in a shaker incubator at 31° C. for 6 days. The conjugates were purified by affinity chromatography using protein A affinity chromatography. Following elution proteins were finally dialyzed against PBS. Purified proteins were characterized for their size and homogeneity by SDS-PAGE and size exclusion chromatography, respectively. SDS-PAGE analysis was performed under reducing and non-reducing conditions on 10% or 12% acrylamide gels and stained using coomassie blue. Size-exclusion chromatography was performed on an AKTA FPLC system using a Superdex 200 increase 10/300GL column.

The IL9 conjugates based on IgG4 (F8(IgG4-HC)-mIL9 and KSF(IgG4-HC)-mIL9) were expressed by transient gene expression in CHO cells. The mammalian expression vector pMM137-F8IgG4-HC-mIL9 and pMM137-KSFIgG4-HC-mIL9, were used for the expression of the F8 (IgG4-HC)-mIL9 and KSF (IgG4-HC)-mIL9 conjugates (FIG. 12A). Each of the two vectors contain 2 mammalian expression cassettes for the independent expression of the light chain (LC) and heavy chain fused to mIL9 (HC-mIL9) of the F8IgG4-HC-mIL9 or KSFIgG4-HC-mIL9 fusion proteins, respectively. For transfection 4×10⁶ cells/mL cells were resuspended in ProCHO-4 Medium supplemented with 4 mM Ultraglutamine. 0.625 μg of plasmid DNAs per million cells followed by 2.5 μg polyethyleneimine per million cells were added to the cells and gently mixed. Transfected cultures were incubated in a shaking incubator at 31° C. with 5% CO2 atmosphere shaking at 120 rpm for 6 days. The fusion proteins were purified by affinity chromatography using protein A agarose beads according to the protocol provided by the supplier. Following elution proteins were dialyzed against PBS. Purified proteins were characterized for their size and homogeneity by SDS-PAGE and size exclusion chromatography, respectively. For SDS-PAGE analysis proteins were run under reducing and non-reducing conditions on 4-12% acrylamide gels and stained using Coomassie blue. Size-exclusion chromatography was performed on an AKTA FPLC system using a Superdex 200 increase 10/300GL column.

1.3 Results

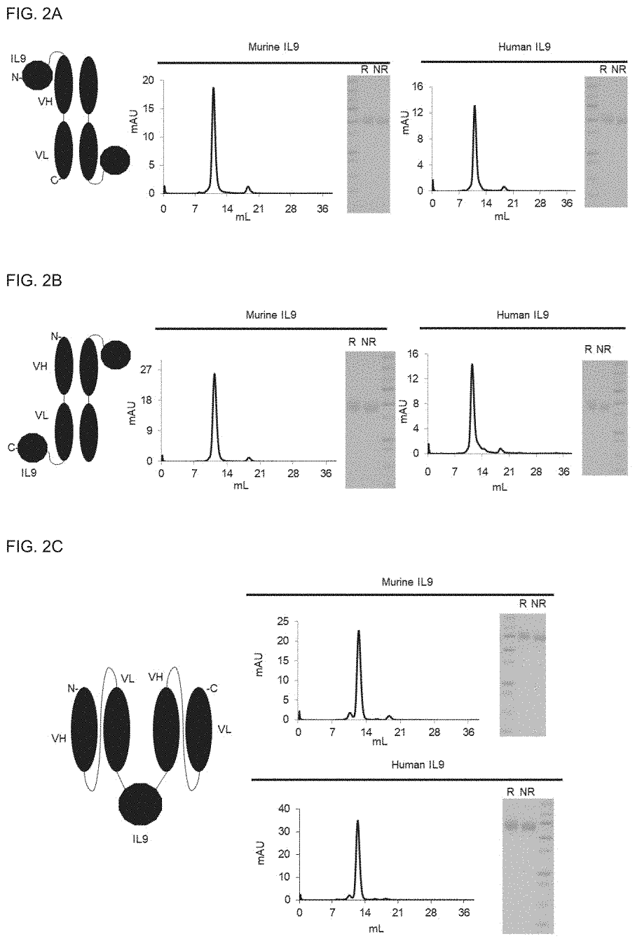

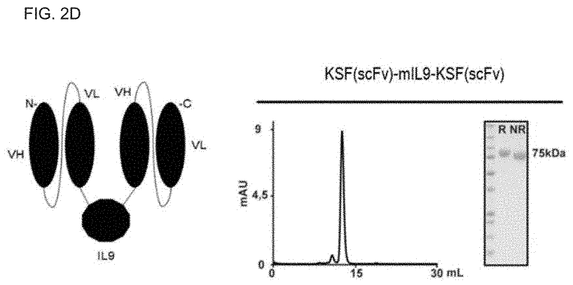

The human IL9 and murine IL9 conjugates were successfully expressed by transient gene expression (TGE) in Chinese hamster ovary (CHO) cells. The purified conjugates exhibited favourable biochemical properties as confirmed using SDS-PAGE and size exclusion chromatography. The presence of single peaks in SEC analysis confirmed the homogeneity of all conjugate preparations (FIGS. 2A-2D, 12B and 12C).

### Example 2: Characterization of Conjugates

2.1 Biacore Analysis

Binding affinity was determined by surface plasmon resonance using a BIAcore X100 instrument using a CM5 chip coated with recombinant fibronectin 11A12 domain. Samples were injected as serial-dilution, using a concentration range from 1 mM to 125 nM.

2.2 In Vitro Bioactivity Assay

The bioactivity of mIL9 conjugates was tested in a proliferation assay using MC/9 cells. Those mast cells derived from mouse liver were expanded in suspension in DMEM, supplemented with 10% FBS, 10% rat T-Stim, 2 mM UltraGlutamine and 0.05 mM—BetamercaptoEtOH. MC/9 cells were seeded at 40,000 cells per well in a 96-well plate in 200 μL of culturing medium (with 5% FBS and 2.5% rat T-Stim). Murine IL9 conjugates and commercial murine IL9 were added to the cultures at various concentrations starting from 400 ng/mL of IL9 equivalent (28 nM). After 70 hours of incubation at 37° C., 20 μL of Cell Titer Aqueous One Solution was added to the wells and the absorption at 492 nm was measured after 2 hours of incubation at 37° C.

2.3 Stability Analysis

The melting temperature of the different mIL9 conjugates was determined using a StepOne real-Time PCR system in combination with the Protein Thermal Shift kit.

Storage stability of F8(scFv)-mIL9-F8(scFv) was assessed by size exclusion chromatography profile and analytical SDS-PAGE analysis of the protein after: a) incubation at 37° C. for 3, 5 and 7 days, b) incubation at 4° C. for 7 days and c) a series of 3 freeze and thaw cycles.

2.4 Results

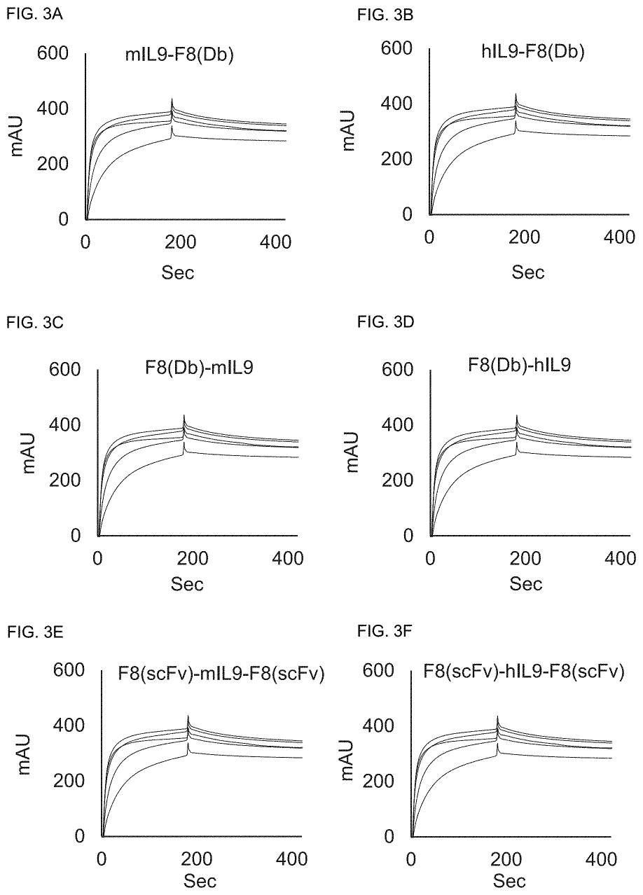

The surface plasmon resonance analysis showed comparable apparent KD for all conjugates tested, in a nM range (FIGS. 3A-3F).

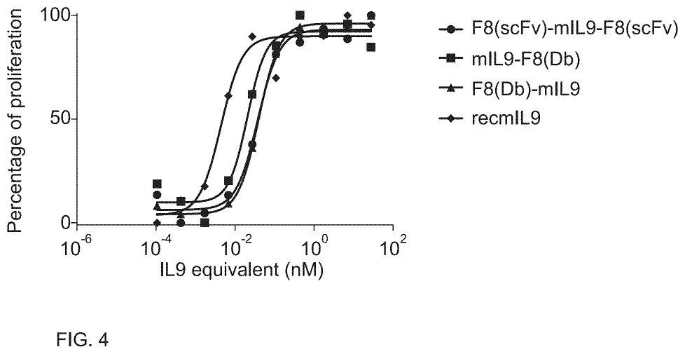

All of the various mIL9 conjugates tested showed biological activity with EC50 values in the range of 0.02 to 0.07 nM for F8(scFv)-mIL9-F8(scFv) and F8(db)-mIL9 and 0.012 to 0.034 nM mIL9-F8(db) in an activity assay (FIG. 4).

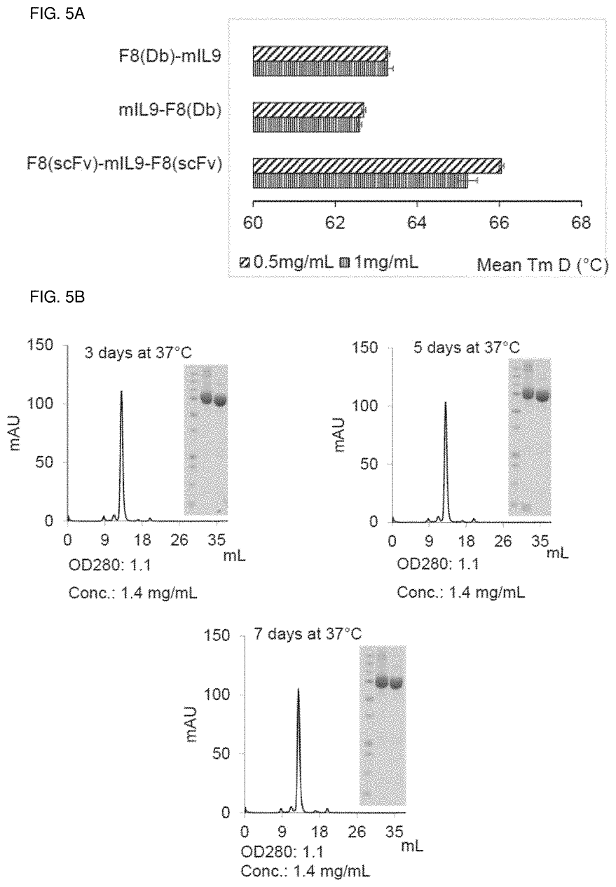

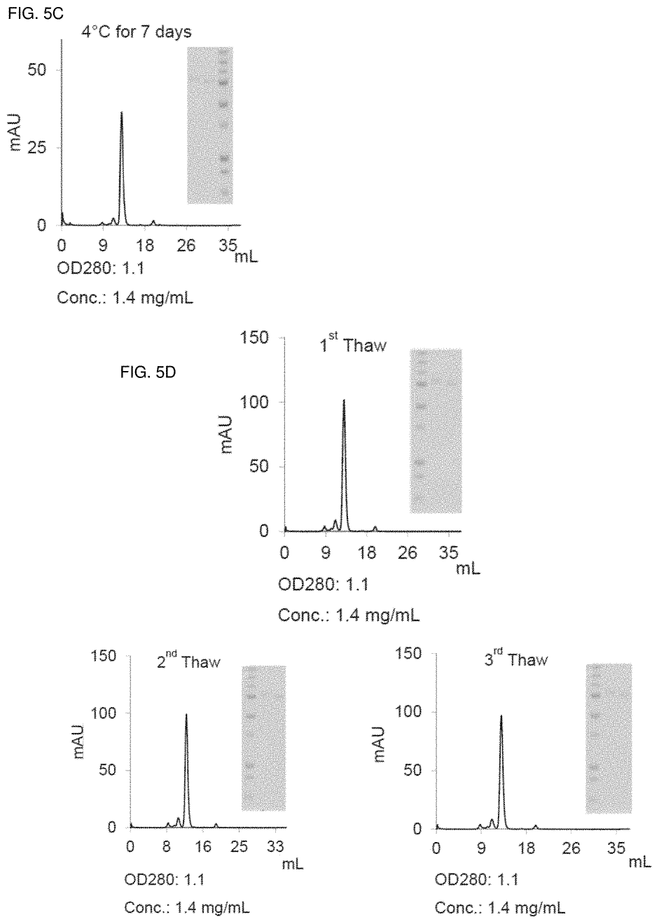

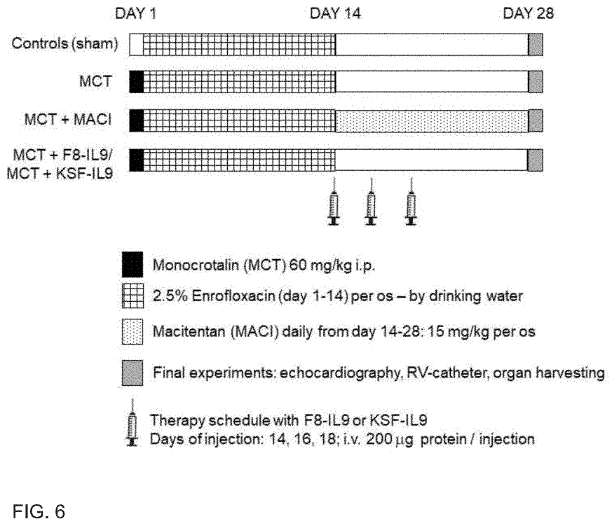

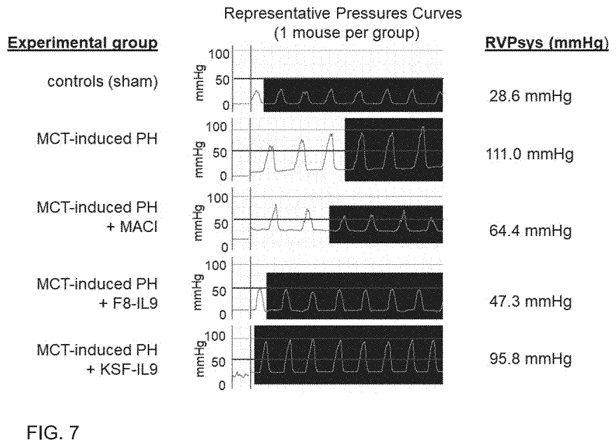

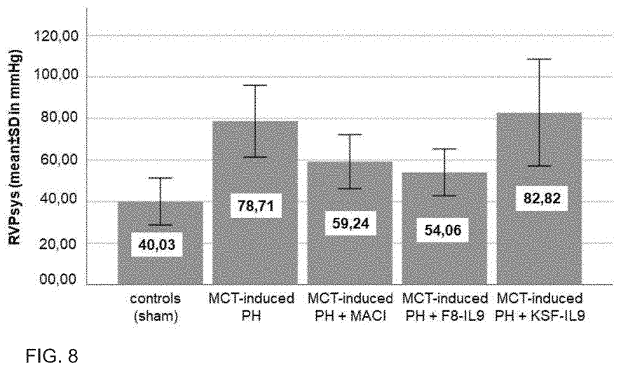

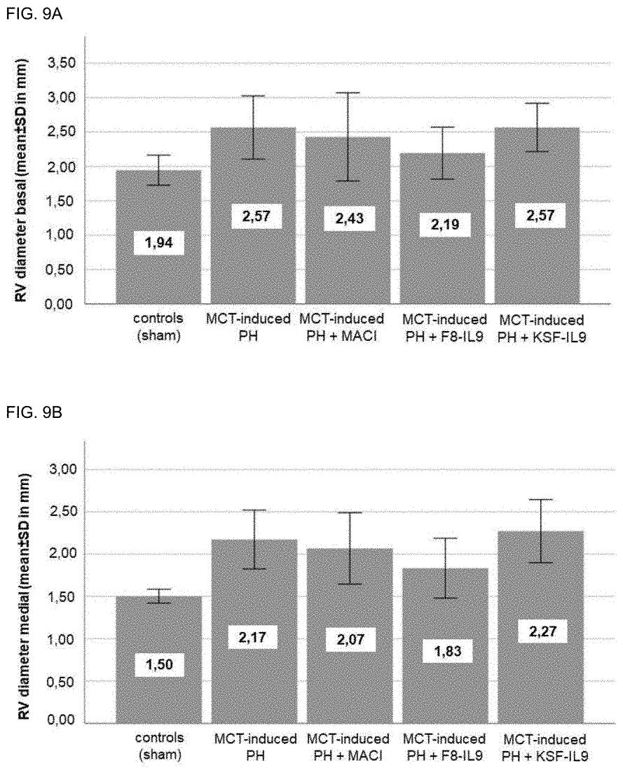

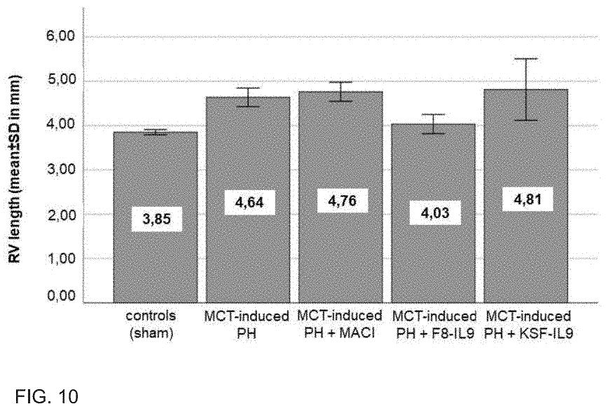

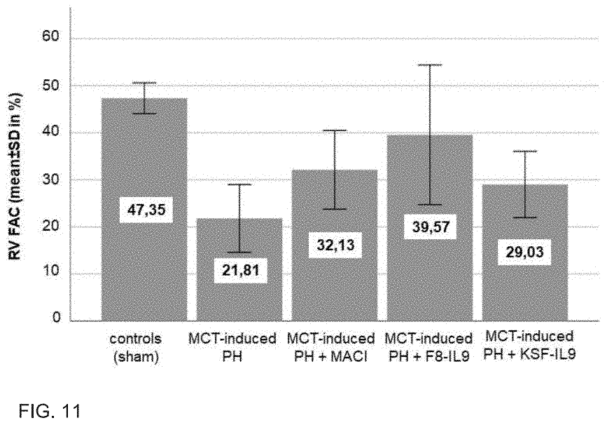

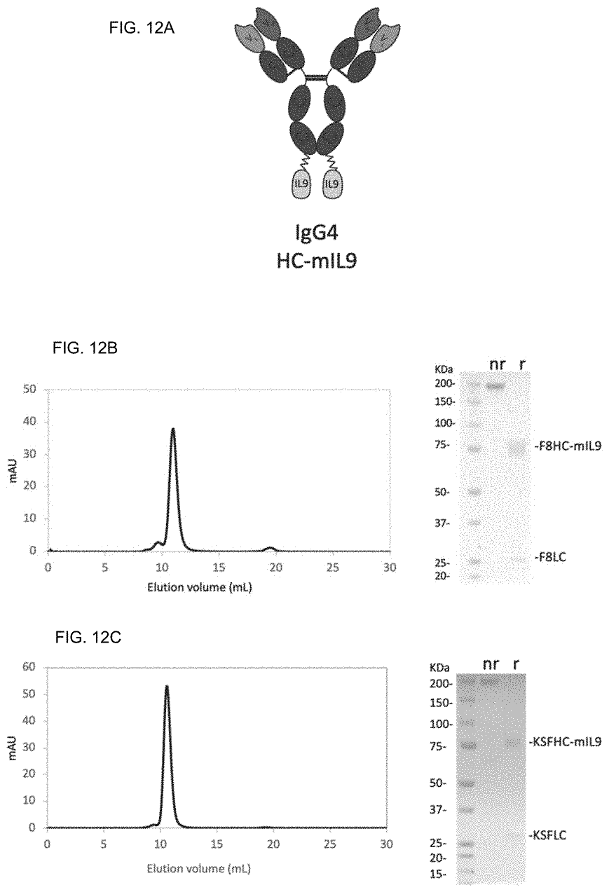

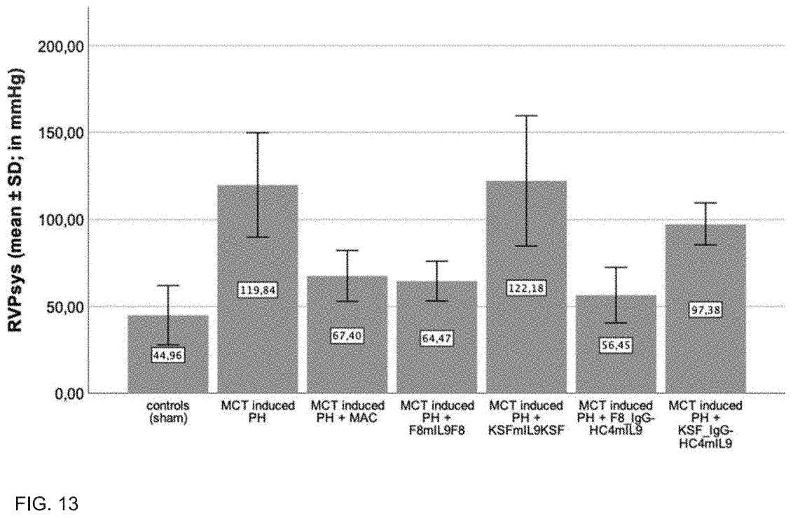

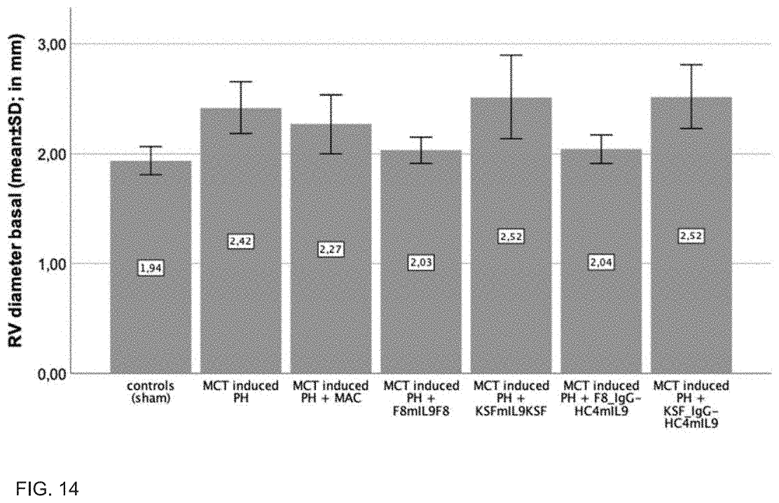

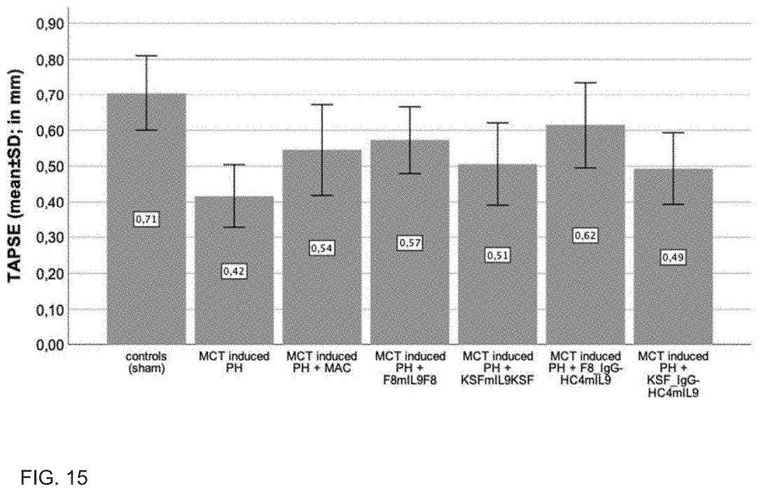

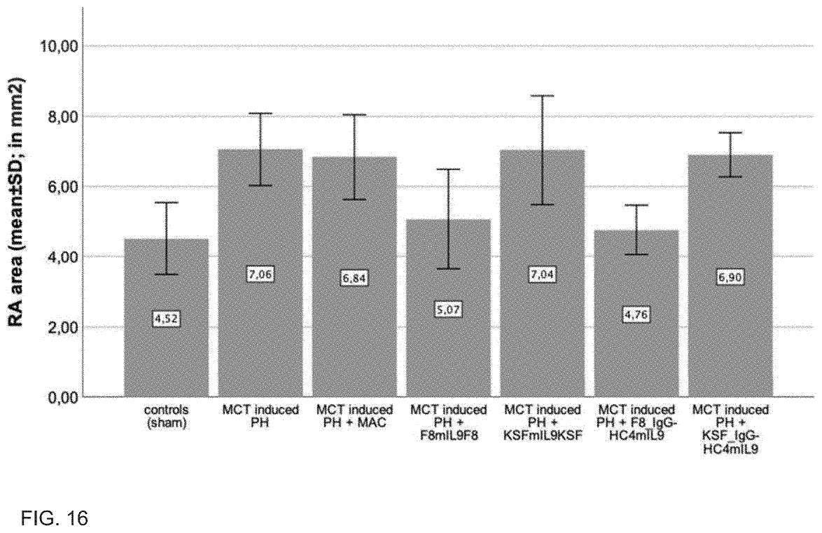

The study of the thermal stability of the different conjugates tested showed that the “Crab” format F8(scFv)-mIL9-F8(scFv) displayed an about 3° C. higher melting temperature compared with the two diabody formats F8(db)-mIL9 and mIL9-F8(db) (FIG. 5A). The F8(scFv)-mIL9-F8(scFv) conjugate also displayed good thermal stability over time with regard to the loss of protein concentration, aggregation, or degradation as assessed by OD280 measurement, SEC analysis and SDS-PAGE analysis. Indeed, when incubated at 37° C. (FIG. 5B) or 4° (FIG. 5C) for a period of up to 7 days or when subjected to 3 cycles of freeze and thawing (FIG. 5D) no loss of concentration, no changes in the size exclusion profile or SDS-PAGE analysis, was detected. Taken together these experiments show that the F8(scFv)-mIL9-F8(scFv) conjugate was more stable than the F8 diabody-based conjugates.

### Example 3: Activity of F8(scFv)-mIL9-F8(scFv) in a Mouse Model of Monocrotaline (MCT) Induced Pulmonary Hypertension (PH)

3.1 Mouse Model of Monocrotaline (MCT) Induced Pulmonary Hypertension (PH) and Treatment Schedule

Pulmonary Hypertension (PH) was induced in C57BL/6 mice (bodyweight: 25-30 g). The animals were obtained from ZET facility (Zentale Experimentelle Tierhaltung) of the University Hospital Jena (UKJ, Jena, Germany). Prior to PH induction, mice were allowed to acclimatize for at least 7 days with ad libitum access to food and water as well as controlled light/dark cycles. As shown in the overview of the experimental design and treatment schedule (FIG. 6), the following experimental groups were investigated:
sham induced controls (n=6)
MCT induced PH (n=8)
MCT induced PH+MACI (n=7)
MCT induced PH+F8(scFv)-mIL9-F8(scFv) (also named F8-IL9) (n=6)
MCT induced PH+KSF(scFv)-mIL9-KSF(scFv) (also named KSF-IL9) (n=6)

For PH induction, the Monocrotaline (MCT) method was used.

The sham induced controls were injected with 30 μl NaCl not containing MCT at day 1 (single dose; intraperitoneally, i.p.). These mice did not develop PH and thus served as healthy controls. The other 4 experimental groups were injected with MCT to induce PH (single dose; intraperitoneally, i.p.; 60 mg/kg body weight; volume 30 μl). Animals in the MCT induced PH+MACI group received the drug (Macitentan) from day 14 to day 28 (once daily; per os; 15 mg/kg body weight). Animals in the MCT induced PH+F8-IL9 group received F8(scFv)-mIL9-F8(scFv) 3 times on day 14, 16 and 18 (intravenously, i.v.; 200 μg/injection; volume 155 μl). Animals in the MCT induced PH+KSF-IL9 group received KSF(scFv)-mIL9-KSF(scFv) 3 times on day 14, 16 and 18 (intravenously, i.v.; 200 μg/injection; volume 135 μl).

To prevent infection and inflammatory alterations of the lungs, mice in all groups received Enrofloxacin (Baytril) 2.5% ad water from days 1 to 14 after MCT injection.

All animals were weighed and examined twice weekly for clinical monitoring of well-being. The clinical condition was assessed using an established score (clinical severity score=CSS) from 1 to 5 (1=no signs of clinical alterations, 2=low-grade impairment, 3=mid-grade impairment, 4=high grade impairment, 5=exitus) obtained by evaluating spontaneous activity, reaction to exogenous stimuli and posture.

All experiments were conducted according to the National Institute of Health Guidelines for the Care and Use of Laboratory Animals (8th edition), to the European Community Council Directive for the Care and Use of Laboratory Animals of 22 Sep. 2010 (2010/63/EU), the current version of the German Law on the Protection of Animals and the guidelines for animal care. The study protocol was approved by the appropriate State Office of Food Safety and Consumer Protection (TLLV, Bad Langensalza, Germany; local registration number: UKJ17-003).

3.2 Echocardiographic Assessment

Echocardiographic assessment was performed on day 28 (FIG. 6) using the Vevo 770 Rodent-Ultrasound-System, Visual Sonic, Canada, 17 MHz probe RMV176. Before echocardiography, mice were anesthetized with isoflurane for a duration time of less than 10 minutes (isoflurane-CP, 2.5V %, FiO2 1.0, oxygen per inhalation-flow dosage). Body temperature and respiratory rate were continuously monitored. All surrogate parameters of right ventricular (RV) morphology and function were assessed, among others, RV basal and medial diameters (in mm), RV length (in mm), and RV fractional area change (FAC, in %).

3.3 Right Heart Catheterization

On day 28 after MCT injection, mice of all experimental groups were deeply anesthetized with a single dose of 100 mg/kg body weight ketamine and 10 mg/kg body weight Xylazin in approximately 60 μl each administered i.p.

Right heart catheterization using a 1.4 F micro conductance pressure-volume catheter (Model SPR-839; Millar Instruments Inc; PowerLab system, ADInstruments Ltd., Oxford, UK) was performed via the right vena jugularis interna to measure the systolic right ventricular pressure and thereby verify the success of the experimental setting. Mice were then sacrificed in deep anesthesia and analgesia to carry out cardiac blood collection after thoracotomy and to harvest the organs.

3.4 Statistics

Statistical analyses were performed using IBM SPSS statistical software, version 25.0 (IBM SPSS Statistics for Windows. Armonk, N.Y., USA). Data are expressed as mean±standard deviation. Mann-Whitney-U test was used to test for significant differences between the different groups.

3.5 Results

The main findings of the echocardiographic and hemodynamic evaluation of the treatment study are presented in FIGS. 7, 9A, 9B, to 11.

Pulmonary Hypertension (PH) could be successfully induced in C57BL/6 mice using the Monocrotaline (MCT) method. The mice exhibited both significantly elevated right ventricular systolic pressure (RVPsys) values and clear signs of right heart overload and dysfunction as assessed by echocardiography compared with sham induced mice. A variety of surrogate markers were used, e.g., diameters and length of the right ventricle as signs of pressure overload, as well as fractional area change as biomarkers of right ventricular dysfunction. Thus, the model used by the inventors excellently qualifies for preclinical studies to evaluate treatment of PH by bioactive payloads.

In this experiment, the potential beneficial effects of a targeted delivery of IL9 using the F8 antibody specific to ED-A were tested and compared to both the KSF antibody, which is of irrelevant specificity in the mouse and therefore qualified as untargeted control, and Macitentan (MACI) as a dual endothelin receptor antagonist proven to effectively reduce pulmonary artery pressures and therefore used as an approved standard therapy also in humans.

In contrast to treatment with KSF(scFv)-mIL9-KSF(scFv), treatment with F8(scFv)-mIL9-F8(scFv) significantly reduced pressure values in the right ventricle as the main pathophysiological surrogate of PH and significantly improved the majority of echocardiographic signs of right ventricular load and dysfunction. The latter are known to determine prognosis in humans suffering from PH. The documented treatment effects of F8(scFv)-mIL9-F8(scFv) in this experiment were at least comparable to the effects achieved by MACI, an established standard therapy for PHA in humans.

### Example 4—Activity of F8(scFv)-mIL9-F8(scFv) (F8-mIL9-F8 in CrAb Format) and F8 IgG-HC4mIL9 in a Mouse Model of Monocrotaline (MCT) Induced Pulmonary Hypertension (PH) 4.1 Mouse Model of Monocrotaline (MCT) Induced Pulmonary Hypertension (PH) and Treatment Schedule

To induce Pulmonary Hypertension (PH), C57BL/6 mice (bodyweight: 25-30 g) were used. The animals were obtained from ZET facility (Zentrale Experimentelle Tierhaltung) of the University Hospital Jena (UKJ, Jena, Germany). Prior to PH induction, mice were allowed to acclimatize for at least 7 days with ad libitum access to food and water as well as controlled light/dark cycles. In this set of experiments, 50 animals were investigated divided into the following 7 experimental groups:
sham induced controls (n=10)
MCT induced PH (n=8)
MCT induced PH+MACI (n=8)
MCT induced PH+F8mIL9F8 in CrAb format (n=6)
MCT induced PH+KSFmIL9KSF in CrAb format (n=6)
MCT induced PH+F8_(IgG-HC4)-mIL9 (n=6)
MCT induced PH+KSF_(IgG-HC4)-mIL9 (n=6)

For PH induction, the Monocrotaline (MCT, Carl Roth, Germany) method was used.

The sham induced controls were injected with 30 μl NaCl not containing MCT at day 1 (single dose; intraperitoneally, i.p.). These mice did not develop PH and thus served as healthy controls. The other 6 experimental groups were injected with MCT (single dose; intraperitoneally, i.p.; 60 mg/kg body weight; volume 30 μl). Animals in the MCT induced PH+MACI group received the drug (Macitentan, Actelion Pharmaceuticals Ltd.) from day 14 to day 28 (once daily; per os; 15 mg/kg body weight).

Animals in the MCT induced PH+F8mIL9F8 in CrAb format group received F8mIL9F8 3 times on days 14, 16 and 18 (intravenously, i.v.; 200 μg/injection; volume 154 μl). Animals in the MCT induced PH+KSFmIL9KSF in CrAb format group received KSFmIL9KSF 3 times on days 14, 16 and 18 (intravenously, i.v.; 198 μg/injection; volume 182 μl).

Animals in the MCT induced PH+F8_(IgG-HC4)-mIL9 group received F8_(IgG-HC4)-mIL9 3 times on days 14, 16 and 18 (intravenously, i.v.; 261 μg/injection; volume 137 μl). Animals in the MCT induced PH+KSF_(IgG-HC4)-mIL9 group received KSF_IgG-HC4mIL9 3 times on days 14, 16 and 18 (intravenously, i.v.; 258 μg/injection; volume 161 μl).

To prevent infection and inflammatory alterations of the lungs, mice in all groups received Enrofloxacin (Baytril, WDT, Germany) 2.5% ad water from days 1 to 14 after MCT injection. All animals were weighed and examined twice weekly for clinical monitoring of well-being. The clinical condition was assessed using an established score (clinical severity score=CSS) from 1 to 5 (1=no signs of clinical alterations, 2=low-grade impairment, 3=mid-grade impairment, 4=high grade impairment, 5=exitus) obtained by evaluating spontaneous activity, reaction to exogenous stimuli and posture.

All experiments were conducted according to the National Institute of Health Guidelines for the Care and Use of Laboratory Animals (8th edition), to the European Community Council Directive for the Care and Use of Laboratory Animals of 22 Sep. 2010 (2010/63/EU), the current version of the German Law on the Protection of Animals and the guidelines for animal care. The study protocol was approved by the appropriate State Office of Food Safety and Consumer Protection (TLLV, Bad Langensalza, Germany; local registration number: UKJ17-003).

4.2 Echocardiographic Assessment

Echocardiographic assessment was performed on day 28 using the Vevo 770 Rodent-Ultrasound-System, Visual Sonic, Canada, 17 MHz probe RMV176. Before echocardiography, mice were anesthetized with isoflurane for a duration time of less than 10 minutes (isoflurane-CP, 2.5V %, FiO2 1.0, oxygen per inhalation-flow dosage). Body temperature and respiratory rate were continuously monitored. All surrogate parameters of right ventricular (RV) morphology and function were assessed, among others, RV basal and medial diameters (in mm), RV length (in mm), tricuspid annular plane systolic excursion (TAPSE, in mm), right atrial area (RA area, in mm²) or main pulmonary artery diameter (MPA, in mm).

4.3 Right Heart Catheterization

On day 28 after MCT injection, mice of all experimental groups were deeply anesthetized with a single dose of 100 mg/kg body weight ketamine and 10 mg/kg body weight Xylazin in approximately 60 μl each administered i.p.

Right heart catheterization using a 1.4 F micro conductance pressure-volume catheter (Model SPR-839; Millar Instruments Inc; PowerLab system, ADInstruments Ltd., Oxford, UK) was performed via the right vena jugularis interna to measure the systolic right ventricular pressure and thereby verify the success of the experimental setting. Mice were then sacrificed in deep anesthesia and analgesia to carry out cardiac blood collection after thoracotomy and to harvest the organs.

4.4 Statistics

Statistical analyses were performed using IBM SPSS statistical software, version 25.0 (IBM SPSS Statistics for Windows. Armonk, N.Y., USA). Data are expressed as mean±standard deviation. Mann-Whitney-U test was used to test for significant differences between the different groups.

4.5. Results

The main findings of the echocardiographic and hemodynamic evaluation of this treatment study are presented in FIGS. 13 to 16.

Pulmonary Hypertension (PH) using the Monocrotaline (MCT) method was successfully induced in C57BL/6 mice, which exhibit both, significantly elevated right ventricular systolic pressure (RVPsys) values, and clear signs of right heart overload and dysfunction as assessed by echocardiography. Here, a variety of surrogate markers demonstrate convincing evidence, e.g., diameters of the right ventricle as signs of pressure overload as well as tricuspid annular plane systolic excursion as biomarker of right ventricular dysfunction or right atrial area shown to be of prognostic relevance in humans. Thus, the model used by us qualifies for preclinical studies to evaluate treatment effects of bioactive payloads.

In this experiment, the potential beneficial effects of a targeted delivery of IL9 using the F8 antibody specific to ED-A⁺ Fn in two different formats (i) F8mIL9F8 in CrAb format and (ii) F8_(IgG-HC4)-mIL9 were tested compared to the corresponding control immunocytokines containing the KSF antibody, which is of irrelevant specificity in the mouse, and Macitentan (MACI) a dual endothelin receptor antagonist proven to effectively reduce pulmonary artery pressures and therefore used as an approved therapy also in humans.

In contrast to the KSF containing control immunocytokines (KSFmIL9KSF in CrAb format and KSF_(IgG-HC4)mIL9), treatment with F8mIL9F8 in CrAb format and F8_(IgG-HC4)-mIL9 significantly reduced pressure values in the right ventricle (RVPsys, in mmHg) as the main pathophysiological surrogate of PH and significantly improved a variety of echocardiographic signs of right ventricular load and dysfunction which are known to relevantly determine prognosis in humans suffering from PH.

SEQUENCE LISTING
Amino acid sequences of the F8 CDRs
F8 CDR1 VH - LFT (SEQ ID NO: 1)
F8 CDR2 VH - SGSGGS (SEQ ID NO: 2)
F8 CDR3 VH - STHLYL (SEQ ID NO: 3)
F8 CDR1 VL - MPF (SEQ ID NO: 4)
F8 CDR2 VL - GASSRAT (SEQ ID NO: 5)
F8 CDR3 VL - MRGRPP (SEQ ID NO: 6)
Amino acid sequence of the F8 VH domain
(SEQ ID NO: 7)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSS
Amino acid sequence of the F8 VL domain
(SEQ ID NO: 8)
EIVLTQSPGTLSLSPGERATLSCRASQSVSMPFLAWYQQKPGQAPRLLIYGASSRATGIPDRFS
GSGSGTDFTLTISRLEPEDFAVYYCQQMRGRPPTFGQGTKVEIK
Amino acid sequence of the F8(scFv)
(SEQ ID NO: 9)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSGGGGSG
GGGSGGGGEIVLTQSPGTLSLSPGERATLSCRASQSVSMPFLAWYQQKPGQAPRLLIYGASS
RATGIPDRFSGSGSGTDFTLTISRLEPEDFAVYYCQQMRGRPPTFGQGTKVEIK
Amino acid sequence of the F8(Db)
(SEQ ID NO: 10)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSGGSGGEI
VLTQSPGTLSLSPGERATLSCRASQSVSMPFLAWYQQKPGQAPRLLIYGASSRATGIPDRFSG
SGSGTDFTLTISRLEPEDFAVYYCQQMRGRPPTFGQGTKVEIK
Amino acid sequence of the KSF VH domain
(SEQ ID NO: 11)
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKSPKVSLFDYWGQGTLVTVSS
Amino acid sequence of the KSF VL domain
(SEQ ID NO: 12)
SSELTQDPAVSVALGQTVRITCQGDSLRSYYASWYQQKPGQAPVLVIYGKNNRPSGIPDRFSG
SSSGNTASLTITGAQAEDEADYYCNSSPLNRLAVVFGGGTKLTVLG
Amino acid sequence of the KSF(scFv)
(SEQ ID NO: 13)
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKSPKVSLFDYWGQGTLVTVSSGGGGS
GGGGSGGGGSSELTQDPAVSVALGQTVRITCQGDSLRSYYASWYQQKPGQAPVLVIYGKNN
RPSGIPDRFSGSSSGNTASLTITGAQAEDEADYYCNSSPLNRLAVVFGGGTKLTVLG
Amino acid sequence of the KSF(Db)
(SEQ ID NO: 14)
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKSPKVSLFDYWGQGTLVTVSSGGSGG
SELTQDPAVSVALGQTVRITCQGDSLRSYYASWYQQKPGQAPVLVIYGKNNRPSGIPDRFSGS
SSGNTASLTITGAQAEDEADYYCNSSPLNRLAVVFGGGTKLTVLG
Amino acid sequence of murine IL9
(SEQ ID NO: 15)
QRCSTTWGIRDTNYLIENLKDDPPSKCSCSGNVTSCLCLSVPTDDCTTPCYREGLLQLTNATQ
KSRLLPVFHRVKRIVEVLKNITCPSFSCEKPCNQTMAGNTLSFLKSLLGTFQKTEMQRQKSRP
Amino acid sequence of human IL9
(SEQ ID NO: 16)
QGCPTLAGILDINFLINKMQEDPASKCHCSANVTSCLCLGIPSDNCTRPCFSERLSQMTNTTMQ
TRYPLIFSRVKKSVEVLKNNKCPYFSCEQPCNQTTAGNALTFLKSLLEIFQKEKMRGMRGKI
Amino acid sequence of F8(scFv)-mIL9-F8(scFv) (″CrAb″ format)
(SEQ ID NO: 17)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSGGGGSG
GGGSGGGGEIVLTQSPGTLSLSPGERATLSCRASQSVSMPFLAWYQQKPGQAPRLLIYGASS
RATGIPDRFSGSGSGTDFTLTISRLEPEDFAVYYCQQMRGRPPTFGQGTKVEIKSGGSGSGGG
GQRCSTTWGIRDTNYLIENLKDDPPSKCSCSGNVTSCLCLSVPTDDCTTPCYREGLLQLTNAT
QKSRLLPVFHRVKRIVEVLKNITCPSFSCEKPCNQTMAGNTLSFLKSLLGTFQKTEMQRQKSRP
SGGSGSGGGGEVQLLESGGGLVQPGGSLRLSCAASGFTFSLFTMSWVRQAPGKGLEWVSAI
SGSGGSTYYADSVKGRFTISRDNSKNTLYLQMNSLRAEDTAVYYCAKSTHLYLFDYWGQGTLV
TVSSGGGGSGGGGSGGGGEIVLTQSPGTLSLSPGERATLSCRASQSVSMPFLAWYQQKPGQ
APRLLIYGASSRATGIPDRFSGSGSGTDFTLTISRLEPEDFAVYYCQQMRGRPPTFGQGTKVEI
K
Amino acid sequence of F8(scFv)-hIL9-F8(scFv) (″CrAb″ format)
(SEQ ID NO: 18)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSGGGGSG
GGGSGGGGEIVLTQSPGTLSLSPGERATLSCRASQSVSMPFLAWYQQKPGQAPRLLIYGASS
RATGIPDRFSGSGSGTDFTLTISRLEPEDFAVYYCQQMRGRPPTFGQGTKVEIKSGGSGSGGG
GQGCPTLAGILDINFLINKMQEDPASKCHCSANVTSCLCLGIPSDNCTRPCFSERLSQMTNTTM
QTRYPLIFSRVKKSVEVLKNNKCPYFSCEQPCNQTTAGNALTFLKSLLEIFQKEKMRGMRGKIS
GGSGSGGGGEVQLLESGGGLVQPGGSLRLSCAASGFTFSLFTMSWVRQAPGKGLEWVSAIS
GSGGSTYYADSVKGRFTISRDNSKNTLYLQMNSLRAEDTAVYYCAKSTHLYLFDYWGQGTLVT
VSSGGGGSGGGGSGGGGEIVLTQSPGTLSLSPGERATLSCRASQSVSMPFLAWYQQKPGQA
PRLLIYGASSRATGIPDRFSGSGSGTDFTLTISRLEPEDFAVYYCQQMRGRPPTFGQGTKVEIK
Amino acid sequence of KSF(scFv)-mIL9-KSF(scFv) (″CrAb″ format)
(SEQ ID NO: 19)
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKSPKVSLFDYWGQGTLVTVSSGGGGS
GGGGSGGGGSSELTQDPAVSVALGQTVRITCQGDSLRSYYASWYQQKPGQAPVLVIYGKNN
RPSGIPDRFSGSSSGNTASLTITGAQAEDEADYYCNSSPLNRLAVVFGGGTKLTVLGSGGSGS
GGGGQRCSTTWGIRDTNYLIENLKDDPPSKCSCSGNVTSCLCLSVPTDDCTTPCYREGLLQLT
NATQKSRLLPVFHRVKRIVEVLKNITCPSFSCEKPCNQTMAGNTLSFLKSLLGTFQKTEMQRQK
SRPSGGSGSGGGGEVQLLESGGGLVQPGGSLRLSCAASGFTFSSYAMSWVRQAPGKGLEW
VSAISGSGGSTYYADSVKGRFTISRDNSKNTLYLQMNSLRAEDTAVYYCAKSPKVSLFDYWGQ
GTLVTVSSGGGGSGGGGSGGGGSSELTQDPAVSVALGQTVRITCQGDSLRSYYASWYQQKP
GQAPVLVIYGKNNRPSGIPDRFSGSSSGNTASLTITGAQAEDEADYYCNSSPLNRLAVVFGGG
TKLTVLG
Amino acid sequence of murine IL9 conjugated to the C-terminus of the F8 diabody (F8(Db)-
mIL9)
(SEQ ID NO: 20)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSGGSGGEI
VLTQSPGTLSLSPGERATLSCRASQSVSMPFLAWYQQKPGQAPRLLIYGASSRATGIPDRFSG
SGSGTDFTLTISRLEPEDFAVYYCQQMRGRPPTFGQGTKVEIKGGGGSGGGGSGGGGSQRC
STTWGIRDTNYLIENLKDDPPSKCSCSGNVTSCLCLSVPTDDCTTPCYREGLLQLTNATQKSRL
LPVFHRVKRIVEVLKNITCPSFSCEKPCNQTMAGNTLSFLKSLLGTFQKTEMQRQKSRP
Amino acid sequence of murine IL9 conjugated to the N-terminus of the F8 diabody (mIL9-
F8(Db)
(SEQ ID NO: 21)
QRCSTTWGIRDTNYLIENLKDDPPSKCSCSGNVTSCLCLSVPTDDCTTPCYREGLLQLTNATQ
KSRLLPVFHRVKRIVEVLKNITCPSFSCEKPCNQTMAGNTLSFLKSLLGTFQKTEMQRQKSRPG
GGGSGGGGSGGGGSEVQLLESGGGLVQPGGSLRLSCAASGFTFSLFTMSWVRQAPGKGLE
WVSAISGSGGSTYYADSVKGRFTISRDNSKNTLYLQMNSLRAEDTAVYYCAKSTHLYLFDYWG
QGTLVTVSSGGSGGEIVLTQSPGTLSLSPGERATLSCRASQSVSMPFLAWYQQKPGQAPRLLI
YGASSRATGIPDRFSGSGSGTDFTLTISRLEPEDFAVYYCQQMRGRPPTFGQGTKVEIK
Amino acid sequence of human IL9 conjugated to the C-terminus of the F8 diabody (F8(Db)-
hIL9)
(SEQ ID NO: 22)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSGGSGGEI
VLTQSPGTLSLSPGERATLSCRASQSVSMPFLAWYQQKPGQAPRLLIYGASSRATGIPDRFSG
SGSGTDFTLTISRLEPEDFAVYYCQQMRGRPPTFGQGTKVEIKGGGGSGGGGSGGGGSQGC
PTLAGILDINFLINKMQEDPASKCHCSANVTSCLCLGIPSDNCTRPCFSERLSQMTNTTMQTRY
PLIFSRVKKSVEVLKNNKCPYFSCEQPCNQTTAGNALTFLKSLLEIFQKEKMRGMRGKI
Amino acid sequence of human IL9 conjugated to the N-terminus of the F8 diabody (hIL9-
F8(Db)
(SEQ ID NO: 23)
QGCPTLAGILDINFLINKMQEDPASKCHCSANVTSCLCLGIPSDNCTRPCFSERLSQMTNTTMQ
TRYPLIFSRVKKSVEVLKNNKCPYFSCEQPCNQTTAGNALTFLKSLLEIFQKEKMRGMRGKIGG
GGSGGGGSGGGGSEVQLLESGGGLVQPGGSLRLSCAASGFTFSLFTMSWVRQAPGKGLEW
VSAISGSGGSTYYADSVKGRFTISRDNSKNTLYLQMNSLRAEDTAVYYCAKSTHLYLFDYWGQ
GTLVTVSSGGSGGEIVLTQSPGTLSLSPGERATLSCRASQSVSMPFLAWYQQKPGQAPRLLIY
GASSRATGIPDRFSGSGSGTDFTLTISRLEPEDFAVYYCQQMRGRPPTFGQGTKVEIK
Amino acid sequence of murine IL9 conjugated to the C-terminus of the KSF diabody (KSF(Db)-
mIL9)
(SEQ ID NO: 24)
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKSPKVSLFDYWGQGTLVTVSSGGSGG
SELTQDPAVSVALGQTVRITCQGDSLRSYYASWYQQKPGQAPVLVIYGKNNRPSGIPDRFSGS
SSGNTASLTITGAQAEDEADYYCNSSPLNRLAVVFGGGTKLTVLGGGGGSGGGGSGGGGSQ
RCSTTWGIRDTNYLIENLKDDPPSKCSCSGNVTSCLCLSVPTDDCTTPCYREGLLQLTNATQKS
RLLPVFHRVKRIVEVLKNITCPSFSCEKPCNQTMAGNTLSFLKSLLGTFQKTEMQRQKSRP
Amino acid sequence of the linker between the VH domain and the VL domain in the F8(scFv)
or in the KSF(scFv)
(SEQ ID NO: 25)
GGGGSGGGGSGGGG
Amino acid sequence of the linker between the VH domain and the VL domain in the F8(Db) or
in the KSF(Db)
(SEQ ID NO: 26)
GGSGG
Amino acid sequence of the linker between murine IL9 or human IL9 and F8(Db), F8(IgG4),
KSF(Db) or KSF (IgG4)
(SEQ ID NO: 27)
GGGGSGGGGSGGGGS
Amino acid sequence of the linker between murine IL9 or human IL9 and the F8(scFv) or the
KSF(scFv) in CrAb format
(SEQ ID NO: 28)
SGGSGSGGGG
Leader sequence-ScFvF8-linker - Forward Primer
(SEQ ID NO: 29)
TCCTCCTGTTCCTCGTCGCTGTGGCTACAGGTGTGCACTCGGAGGTGCAGCTGTTGGAGT
CTGGG
Leader sequence-ScFvF8-linker - Backward Primer
(SEQ ID NO: 30)
ACCTCCACCGCCAGAACCACTTCCGCCTGATTTGATTTCCACCTTGGTCCCTTG
Linker - hIL9-Linker - Forward Primer
(SEQ ID NO: 31)
tcaggcggaagtggttctggcggtggaggtCAGGGGTGTCCAACCTTGGCGGGG
Linker - hIL9-Linker - Backward Primer and HindllI-Leader sequence-ScFvF8-Linker-IL9-Linker-
Backward Primer
(SEQ ID NO: 32)
cccctgaaccactgcctccagaTATCTTGCCT
Linker-mIL9-Linker - Forward Primer
(SEQ ID NO: 33)
tcaggcggaagtggttctggcggtggaggtCAGAGATGCAGCACCACATGGGG
Linker-mIL9-Linker - Backward Primer and Hindlll-Leader sequence-ScFvF8-Linker-mIL9-Linker-
back ward
(SEQ ID NO: 34)
ccccctgaaccactgcctccagaTGGTCGGCTT
Linker-ScFv8-STOP-Notl - Forward Primer
(SEQ ID NO: 35)
gcagtggttcagggggcggtggaGAGGTGCAGCTGT
Linker-ScFv8-STOP-Notl - Backward Primer and Linker-IL9-Linker-ScFv8-STOP-Notl -
Backward Primer
(SEQ ID NO: 36)
TTTTCCTTTTGCGGCCGCTTATTTGATTTCCACCTTGGTCCC
HindlII-Leader sequence-ScFvF8-Linker-IL9-Linker - Forward Primer
(SEQ ID NO: 37)
CCCAAGCTTGTCGACCATGGGCTGGAGCCTGATCCTCCTGTTCCTCGTCGCTGTGGC
Linker-hIL9-Linker-ScFv8-STOP-Notl - Forward Primer
(SEQ ID NO: 38)
CAGGGGTGTCCAACCTTGGCGGGGA
Linker-mIL9-Linker-ScFv8-STOP-Notl - Forward Primer
(SEQ ID NO: 39)
CAGAGATGCAGCACCACATGGGGA
HindIII-Leader sequence -DbF8 - Forward Primer; Hindlll-Leader sequence -IL9 - Forward
primer; Hindlll-Leader sequence -KSF(VH) - Forward Primer and Hindlll-Leader sequence -
DbKSF - forward primer
(SEQ ID NO: 40)
ATTAAAGCTTCCACCATGGGCTGGAGCCTGATCCTCCTGTTCCTCGTCGCTGTGGC
HindlII-Leader sequence -DbF8 - backward primer
(SEQ ID NO: 41)
GCCAGAGCCACCTCCGCCTGAACCGCCTCCACCTTTGATTTCCACCTTGGTCCC
hIL9-Stop-Notl - Forward primer
(SEQ ID NO: 42)
CAGGCGGAGGTGGCTCTGGCGGTGGCGGATCACAGGGGTGTCCAACCTTG
hIL9-Stop-Notl - Backward primer
(SEQ ID NO: 43)
TAATGCGGCCGCTTATCATATCTTGCCTCTCATCCCTCT
mIL9-Stop-Notl - Forward primer
(SEQ ID NO: 44)
CAGGCGGAGGTGGCTCTGGCGGTGGCGGATCACAGAGATGCAGCACCACA
mIL9-Stop-Notl - Backward Primer
(SEQ ID NO: 45)
TAATGCGGCCGCTTATCATGGTCGGCTTTTCTGCCTTTGCAT
Leader sequence-hIL9 - Forward Primer
(SEQ ID NO: 46)
TCCTCCTGTTCCTCGTCGCTGTGGCTACAGGTGTGCACTCGCAGGGGTGTCCAACCTTG
Leader sequence-hIL9 - Backward Primer
(SEQ ID NO: 47)
GCCAGAGCCACCTCCGCCTGAACCGCCTCCACCTATCTTGCCTCTCATCCCTCT
Leader sequence-mIL9 - Forward Primer
(SEQ ID NO: 48)
TCCTCCTGTTCCTCGTCGCTGTGGCTACAGGTGTGCACTCGCAGAGATGCAGCACCACA
Leader sequence-mIL9 - Backward Primer and Hindlll-Leader sequence -mIL9 - Backward
Primer
(SEQ ID NO: 49)
GCCAGAGCCACCTCCGCCTGAACCGCCTCCACCTGGTCGGCTTTTCTGCCTTTGCAT
DbF8-Stop-Notl - Forward Primer
(SEQ ID NO: 50)
CAGGCGGAGGTGGCTCTGGCGGTGGCGGATCAGAGGTGCAGCTGTTGGAGTCT
DbF8-Stop-Notl - Backward Primer
(SEQ ID NO: 51)
TAATGCGGCCGCTTATCATTTGATTTCCACCTTGGTCCC
Hindlll-Leader sequence -KSF(VH) - Backward Primer
(SEQ ID NO: 52)
ACCGCCAGAGCCACCTCCGCCTGAACCGCCTCCACCACTCGAGACGGTGACCAGGG
KSF(VL)-linker - Forward Primer
(SEQ ID NO: 53)
AGGCGGTTCAGGCGGAGGTGGCTCTGGCGGTGGCGGATCGTCTGAGCTGACTCAGGA
KSF(VL)-linker - Backward Primer
(SEQ ID NO: 54)
CTCTGacctccaccgccagaaccacttccgcctgaGCCTAGGACGGTCAGCTTGGT
linker-mIL9-KSF(ScFv)-Notl - Forward Primer
(SEQ ID NO: 55)
ATGGGGAATTCGAGACACCAATTACCTTATTGAA
linker-mIL9-KSF(ScFv)-Notl - Backward Primer
(SEQ ID NO: 56)
taatGCGGCCGCTTATCATCCCAGCACTGTCAGCTT
Hindlll-Leader sequence -DbKSF - Backward Primer
(SEQ ID NO: 57)
AATCGGCTGGCTGTGGTATTCGGCGGAGGGACCAAGCTGACCGTCCTAGGCGGTGGAGG
CGGTTCAGGCGGAGGTGGCTCTGGC
Fragment-A sequence - Forward Primer
(SEQ ID NO: 58)
TTTGACTACTGGGGCCAGGGAACCCTGGTCACCGTCTC
Fragment-A sequence - Backward Primer
(SEQ ID NO: 59)
CCACCGCCAGAGCCACCTCCGCCTGAACCGCCTCCACCACCCAGAGACAGGGAGAGGCT
CTTC
Amino acid sequence of the F8 heavy chain
(SEQ ID NO: 60)

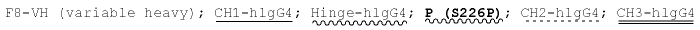

EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSASTKGPS
VFPLAPCSRSTSESTAALGCLVKDYFPEPVTVSWNSGALTSGVHTFPAVLQSSGLYSLSSVVT

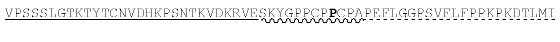

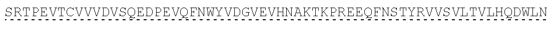

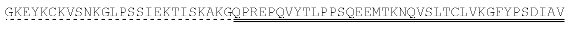

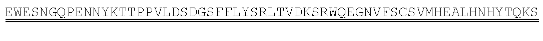

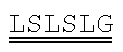

Amino acid sequence of the F8 light chain in the F8 (lgG4-HC)-mIL9 and F8 (IgG4-HC)-hIL9
conjugates
(SEQ ID NO: 61)
F8-VL (variable light); F8-CLk (Constant light-kappa)
EIVLTQSPGTLSLSPGERATLSCRASQSVSMPFLAWYQQKPGQAPRLLIYGASSRATGIPDRFS
GSGSGTDFTLTISRLEPEDFAVYYCQQMRGRPPTFGQGTKVEIKRTVAAPSVFIFPPSDEQLKS
GTASVVCLLNNFYPREAKVQWKVDNALQSGNSQESVTEQDSKDSTYSLSSTLTLSKADYEKH
KVYACEVTHQGLSSPVTKSFNRGEC
Amino acid sequence of F8 heavy chain-mIL9 in the F8 (lgG4-HC)-mIL9 conjugate
(SEQ ID NO: 62)

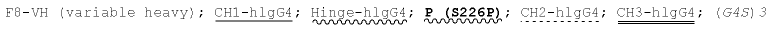

linker; murine IL9 (mIL9)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSASTKGPS
VFPLAPCSRSTSESTAALGCLVKDYFPEPVTVSWNSGALTSGVHTFPAVLQSSGLYSLSSVVT

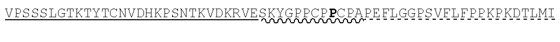

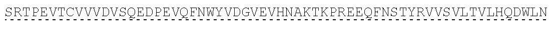

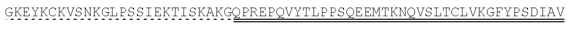

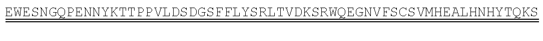

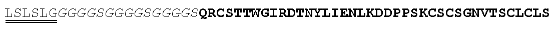

VPTDDCTTPCYREGLLQLTNATQKSRLLPVFHRVKRIVEVLKNITCPSFSCEKPCNQTMAGNT
LSFLKSLLGTFQKTEMQRQKSRP
Amino acid sequence of F8 heavy chain-huIL9 in the F8 (lgG4-HC)-hIL9 conjugate
(SEQ ID NO: 63)

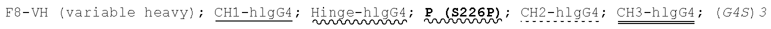

linker; human IL9 (hIL9)
EVQLLESGGGLVQPGGSLRLSCAASGFTFSLFTMSWVRQAPGKGLEWVSAISGSGGSTYYAD
SVKGRFTISRDNSKNTLYLQMNSLRAEDTAVYYCAKSTHLYLFDYWGQGTLVTVSSASTKGPS
VFPLAPCSRSTSESTAALGCLVKDYFPEPVTVSWNSGALTSGVHTFPAVLQSSGLYSLSSVVT

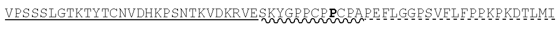

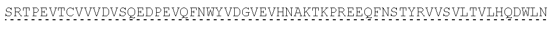

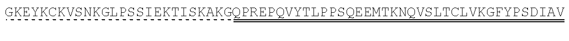

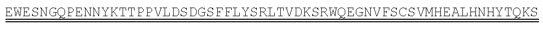

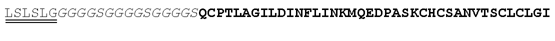

PSDNCTRPCFSERLSQMTNTTMQTRYPLIFSRVKKSVEVLKNNKCPYFSCEQPCNQTTAGNA
LTFLKSLLEIFQKEKMRGMRGKI
Amino acid sequence of KSF light chain in KSF (lgG4-HC)-mIL9 and KSF (lgG4-HC)-hIL9
conjugates
(SEQ ID NO: 64)
KSF-VL (variable light); KSF-CLλ (Constant light-lambda)
SSELTQDPAVSVALGQTVRITCQGDSLRSYYASWYQQKPGQAPVLVIYGKNNRPSGIPDRFSG
SSSGNTASLTITGAQAEDEADYYCNSSPLNRLAVVFGGGTKLTVLGQPKAAPSVTLFPPSSEEL
QANKATLVCLISDFYPGAVTVAWKADSSPVKAGVETTTPSKQSNNKYAASSYLSLTPEQWKSH
KSYSCQVTHEGSTVEKTVAPTECS
Amino acid sequence of KSF heavy chain-mIL9 in the KSF (IgG4-HC)-mIL9 conjugate
(SEQ ID NO: 65)

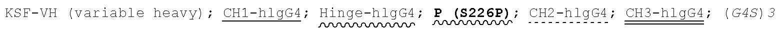

linker; murine IL9 (mIL9)
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKSPKVSLFDYWGQGTLVTVSSASTKGP
SVFPLAPCSRSTSESTAALGCLVKDYFPEPVTVSWNSGALTSGVHTFPAVLQSSGLYSLSSVV

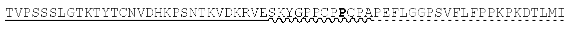

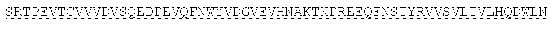

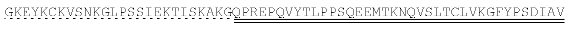

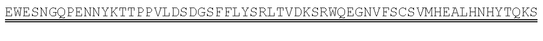

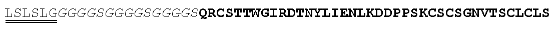

VPTDDCTTPCYREGLLQLTNATQKSRLLPVFHRVKRIVEVLKNITCPSFSCEKPCNQTMAGNT
LSFLKSLLGTFQKTEMQRQKSRP
Amino acid sequence of KSF heavy chain-hIL9 in the KSF (IgG4-HC)-hIL9 conjugate
(SEQ ID NO: 66)

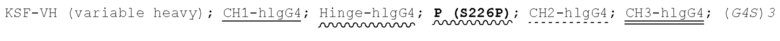

linker; human IL9 (hIL9)
EVQLLESGGGLVQPGGSLRLSCAASGFTFSSYAMSWVRQAPGKGLEWVSAISGSGGSTYYA
DSVKGRFTISRDNSKNTLYLQMNSLRAEDTAVYYCAKSPKVSLFDYWGQGTLVTVSSASTKGP
SVFPLAPCSRSTSESTAALGCLVKDYFPEPVTVSWNSGALTSGVHTFPAVLQSSGLYSLSSVV

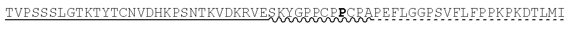

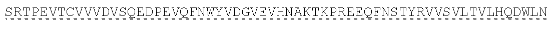

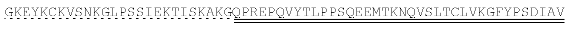

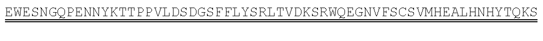

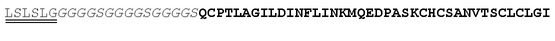

PSDNCTRPCFSERLSQMTNTTMQTRYPLIFSRVKKSVEVLKNNKCPYFSCEQPCNQTTAGNA
LTFLKSLLEIFQKEKMRGMRGKI

1. A conjugate for use in a method for treatment of pulmonary hypertension in a patient, the conjugate comprising interleukin-9 (IL9) and a specific binding member which binds an antigen associated with tissue and/or vascular remodelling.

2. A conjugate for use in a method of delivering IL9 to sites of remodelled lung and/or heart tissue or vasculature in a patient with pulmonary hypertension, the conjugate comprising IL9 and a specific binding member which binds an antigen associated with tissue and/or vascular remodelling.

3. A method of treating pulmonary hypertension in a patient, the method comprising administering to the patient a therapeutically effective amount of a conjugate comprising IL9 and a specific binding member which binds an antigen associated with tissue and/or vascular remodelling.

4. A method of delivering IL9 to sites of remodelled lung and/or heart tissue or vasculature in a patient with pulmonary hypertension, the method comprising administering a conjugate comprising IL9 and a specific binding member which binds an antigen associated with tissue and/or vascular remodelling to the patient.

5. The conjugate for use, or method, according to any one of the preceding claims, wherein the antigen associated with tissue and/or vascular remodelling is the Extra Domain-A of fibronectin.

6. The conjugate for use, or method, according to any one of the preceding claims, wherein the specific binding member is an antibody molecule or an antigen-binding fragment thereof.

7. The conjugate for use, or method, according to any one of claims 1 to 6, wherein the specific binding member is a single-chain Fv (scFv).

8. The conjugate for use, or method, according to any one of claims 1 to 6, wherein the specific binding member is an immunoglobulin G (IgG) molecule, preferably an IgG4.

9. A conjugate comprising interleukin-9 (IL9), wherein the N-terminus of IL9 is conjugated to a first single-chain Fv (scFv) which binds the Extra Domain-A (ED-A) of fibronectin and the C-terminus of IL9 is conjugated to a second scFv which binds the ED-A of fibronectin.

10. A conjugate comprising an IgG, preferably an IgG4, which binds ED-A and two IL9 moieties, wherein the N-terminus of each IL9 is conjugated to the C-terminus of one of the two IgG heavy chains.

11. The conjugate according to any one of the preceding claims, wherein the specific binding member, scFv or IgG comprises an antigen-binding site having the complementarity determining regions (CDRs) of antibody F8 set forth in SEQ ID NOs 1-6.

12. The conjugate according to any one of the preceding claims, wherein the specific binding member, scFv or IgG comprises the VH and VL domains of antibody F8 set forth in SEQ ID NOs 7 and 8.

13. The conjugate according to any one of claims 7, 9, and 11 to 12, wherein the scFv comprises the amino acid sequence of scFv F8 set forth in SEQ ID NO: 9.

14. The conjugate according to any one of claims 8, 10, and 11 to 12, wherein the IgG comprises the amino acid sequence of the F8 heavy chain set forth in SEQ ID NO: 60 and the amino acid sequence of the F8 light chain set forth in SEQ ID NO: 61.

15. The conjugate, conjugate for use, or method, according to any one of the preceding claims wherein the IL9 is human IL9.

16. The conjugate, conjugate for use, or method, according to claim 15, wherein the IL9 comprises the amino acid sequence set forth in SEQ ID NO: 16.

17. The conjugate according to any one of claims 9, 11 to 13 and 15 to 16, wherein the conjugate comprises or consists of the sequence set forth in SEQ ID NO: 18.

18. The conjugate for use, or method, according to any of claims 1 to 7, wherein the conjugate is a conjugate according to any one of claims 9, 11 to 13 and 15 to 17.

19. The conjugate according to any one of claims 10 to 12 and 14 to 16, wherein the conjugate comprises or consists of the sequences set forth in SEQ ID NOs: 61 and 63.

20. The conjugate for use, or method, according to any of claims 1 to 6 and 8, wherein the conjugate is a conjugate according to any one of claims 10 to 12, 14 to 16 and 19.
